(19) United States
(12) Patent Application Publication
(10) Pub. No.: US 2010/0113747 A1
(43) Pub. Date: May 6, 2010

(54) Coupling of Antibody Polypeptides at the C-Terminus

(75) Inventors: Kristian Kjaergaard, Ballerup (DK); Bernd Peschke, Malov (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, Bagsvaerd (DK)

(21) Appl. No.: 12/528,905

(22) PCT Filed: Feb. 8, 2008
(86) PCT No.: PCT/EP2008/051540

§ 371 Date: Nov. 6, 2009

(30) Foreign Application Priority Data
Mar. 5, 2007 (EP) 07103517.4

(51) Int. Cl. C07K 16/00

(52) U.S. Cl. 530/387.3

(57) ABSTRACT

The present invention relates to a process for dimerization of antibody fragments, antibody fragment dimers, pharmaceutical compositions comprising antibody fragment dimers as well as their use in medicaments for therapeutic applications. The methods described can advantageously be used for producing bispecific antibodies and/or bispecific fragments thereof.

### FIELD OF THE INVENTION

The present invention relates to the field of protein chemistry, in particular to dimerization of antibody fragments.

### BACKGROUND OF THE INVENTION

Bispecific antibodies, with affinity towards two independent antigens, have been previously described (reviewed by Holliger and Winter 1993 Curr. Opin. Biotech. 4, 446-449 (see also Poljak, R. J., et al. (1994) Structure 2:1121-1123; Cao et al. (1998), Bioconjugate Chem. 9, 635-644); Aramwit et al. Drugs of the Future 2005, 30, 1013-1016; Moosmayer et al. Clin. Cancer Res. 2006, 12, 5587-5595). Such antibodies may be particularly useful in (among other things) redirection of cytotoxic agents or immune effector cells to target sites, such as tumors. To date, most bispecific antibodies have been created by connecting VH and VL domains of two independent antibodies using a linker that is too short to allow pairing between domains on the same chain, thus driving the pairing between complementary domains on different chains to recreate the two antigen-binding sites. This type of antibody molecule lack the Fc domain and thus the ability of the antibody to trigger an effector function (e.g. complement activation, Fc-receptor binding etc.), and due to its relatively small size, the half life is typically low. A bispecific antibody should contain at least the antigen-binding parts of two antibodies with different specificity, and both parts may be expressed recombinantly. For example, Albrecht et al. (Bioconjugate Chem. 2004, 15, 16-26.) described dimerized ScFvs, which were dimerized via a dithio-linkage.

Bifunctional molecules which have been used as spacers for covalent conjugation of two biomolecules have been described by Li et al. (Bioorg. Med. Chem. Lett, 2005, 15, 5558-5561). However, for preparation of bi-specific antibody constructs, whatever method is used, the linking of the two different antigen-binding parts is a key issue in the preparation. A random dimerization will usually result in mixtures of many different coupling products, being difficult to separate. Existing methods for controlling the linking of fragments reacting with each other are, for example knob-in-hole mutations (Carter, J. Immunol. Methods 2001, 248, 7-15.), leucine-zippers (Kostelny, et al. J. Immunol. 1992, 148, 1547-1553.), or oligonucleotide pairing (Chaudri et al. FEBS Letters, 1999, 450, 23-26.). Constructs in which the heavy and light chain antibody variable domains from two antibodies of different specificity are fused together as a single polypeptide chain have also been described (Kipriyanov and Le Gall, Current Opinion in Drug Discovery & Development, 2004, 7, 233-242.). For the preparation of hetero-dimeric constructs, a principal problem is to control which monomers are going to form the dimer. To the author's knowledge, no methods exist in which the dimerization is controlled by the chemical reaction alone.

Thus, there is a need for improved and alternative processes for producing dimerized antibody constructs such as, e.g., bispecific antibodies or fragments thereof, which can be obtained in commercially relevant yields and which are amenable to purification.

### SUMMARY OF THE INVENTION

In order to overcome the above-mentioned limitations of the known methods for dimerizing antibody fragments, the present invention now provides a process for dimerization of two antibody fragments at the respective heavy chain (HC) C-terminus comprising modification of the C-termini and reacting the C-termini to form a covalent linkage between the two antibody fragments. Also provided by the present invention is a compound comprising a dimer of two antibody fragments, wherein said antibody fragments are coupled at their C-termini of the heavy chain (HC) polypeptides. Also provided by the present invention is an antibody fragment wherein the C-terminus of the HC polypeptide is modified according to the invention.

According to the process provided by the present invention, one antibody fragment bears one chemical functional group, which is not present in the second antibody fragment, and the second antibody fragment bears another chemical group, which is not present in the first antibody fragment. Dimerization can be obtained when these two chemical groups react which each other, leading to a chemical bond.

In a particular embodiment, outlined in FIG. 1, the antibody dimerized fragments are Fab-fragments, each comprising at least the variable domain of a HC associated with a light chain (LC). The C-termini of the HC polypeptides are then linked to form a Fab2 fragment.

The process of the invention has shown useful for producing dimerized Fab-fragments in high yields and purity, as well as allowing both of the constituent Fab-fragments to retain intact N-termini. Similar principles can be applied to dimerization of other antibody fragments at the C-termini of HC polypeptides.

In one aspect, the present invention provides a process for dimerization of two anti-body fragments comprising the steps of

(a) introducing a first chemical group to the C-terminus of the first antibody fragment,

(b) introducing a second chemical group to the C-terminus of the second antibody fragment, and

(c) reacting the first chemical group with the second chemical group to form a covalent linkage of the two antibody fragments.

In another aspect, the present invention provides a process for dimerization of two antibody fragments of antibodies comprising the steps of

(a′) introducing a first chemical group to the C-terminus of the first antibody fragment by reaction with an enzyme in the presence of a nucleophile,

(b′) introducing a second chemical group to the C-terminus of the second antibody fragment by reaction with an enzyme in the presence of a nucleophile, and

(c) reacting the first chemical group with the second chemical group to form a covalent linkage of the two antibody fragments.

In another aspect, the present invention provides a process for dimerization of two antibody fragments of antibodies comprising the steps of

(a′) introducing a first chemical group to the C-terminus of the first antibody fragment by reaction with an enzyme in the presence of a nucleophile,

(b″) introducing a third chemical group to the C-terminus of the second antibody fragment by reaction with an enzyme in the presence of a nucleophile,

(b′″) reacting the third chemical group with a molecule bearing a fourth and a sec- and chemical group to attach said molecule covalently to the C-terminus of the second anti-body fragment by reaction of the third chemical group and the fourth chemical group, and

(c) reacting the first chemical group with the second chemical group to form a covalent linkage of the two antibody fragments.

In one embodiment of each process described above, each antibody fragment is a Fab-fragment.

In another embodiment of each process and embodiment described above, the anti-body fragments have different binding specificities, binding, e.g., different antigens or different epitopes of the same antigen.

In another embodiment of each process and embodiment described above, the C-terminal amino acid sequence of the HC polypeptide of at least one, optionally both, of the antibody fragments is -Leu-Leu-Ala.

In another aspect, the enzyme-catalyzed modification is performed by a serine-protease. In one embodiment, the enzyme-catalyzed modification is performed by a serine-carboxypeptidase. In another embodiment, the enzyme-catalyzed modification is performed by the enzyme carboxypeptidase Y.

In another aspect the present invention relates to an antibody fragment comprising a HC polypeptide wherein the C-terminal amino acid sequence is Leu-Leu-Ala, as well as its use in a process described above. In one embodiment, the antibody fragment is a Fab-fragment.

### DESCRIPTION OF THE DRAWINGS

FIG. 1 outlines a process for dimerizing antibody fragments according to the invention as applied to dimerization of Fab-fragments.

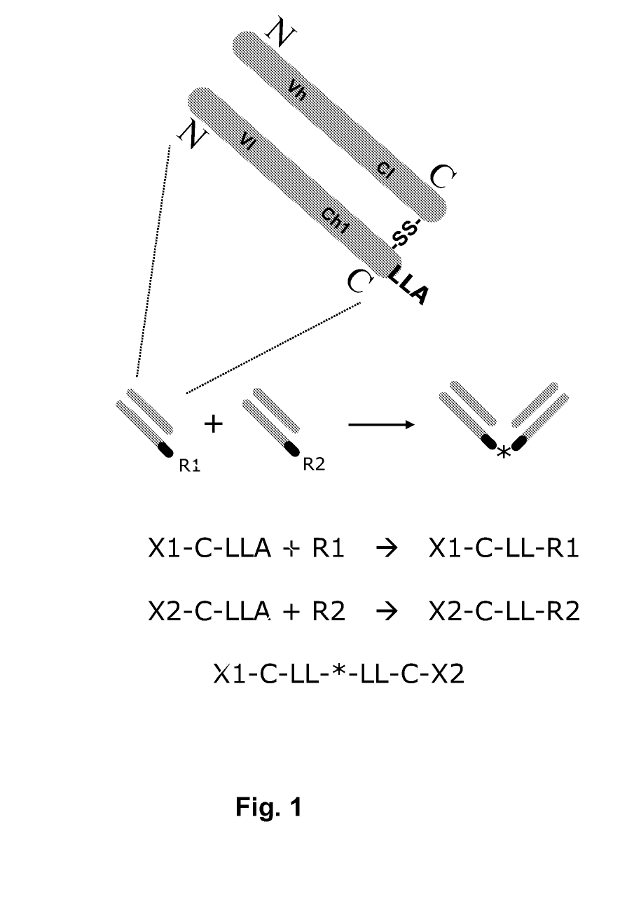

FIG. 2 shows unreduced (A) and reduced (B) SDS-gel analyses of dimerized Fab-fragment preparations as described in Example 1, step 3. The unreduced SDS-gel shows in well 3 and 4 the starting Fab-fragment in two concentrations. Wells 5-12 show the reaction mixture during dimerization (step 3) at different reaction times, each in two concentrations. The reduced SDS-gel shows, in well 2, the starting Fab-fragment, and in well 3 the reaction product after step 3.

### DESCRIPTION OF THE INVENTION

The present invention provides a compound being a dimer of two antibody fragments, wherein said antibody fragments are coupled at their C-termini of the heavy chain (HC) polypeptides.

In one embodiment the compound comprises antibody fragments which are coupled by a non-peptide bond. In another embodiment the antibody fragments being dimerized are Fab-fragments. In another embodiment, the C-terminus of a first HC polypeptide has the structure of

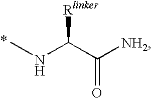

wherein the first polypeptide is marked with “*”, and a second HC-polypeptide is attached to the group R^{linker}. In a particular embodiment, the C-terminus of the first HC-polypeptide has the structure of

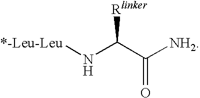

In yet another embodiment the compound comprises antibody fragments which are coupled by a reaction between an azide on one of the antibody fragments and an alkyne on the other antibody fragment. In yet another embodiment the compound comprises antibody fragments which are coupled by a reaction between an O-alkylated hydroxylamine on one of the antibody fragments and a ketone or an aldehyde on the other antibody fragment. In yet another embodiment, the antibody fragments each comprises a HC polypeptide comprising at least two, or all three complementarity-determining regions (CDR) of an antibody.

The present invention also provides an antibody fragments advantageously used in a dimerization process as described herein. In one embodiment, the C-terminus of a HC-polypeptide comprised in the antibody fragment has the structure of

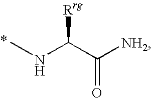

wherein the HC polypeptide is marked with “*” and R^{rg} is a group comprising or bearing a group selected from azide, alkyne, O-alkylated hydroxylamine, ketone, aldehyde, 1,2-diol, or 1,2 aminoalcohol. In one embodiment, the C-terminus of the HC-polypeptide has the structure of

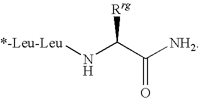

In another embodiment, —R^{rg} is selected from

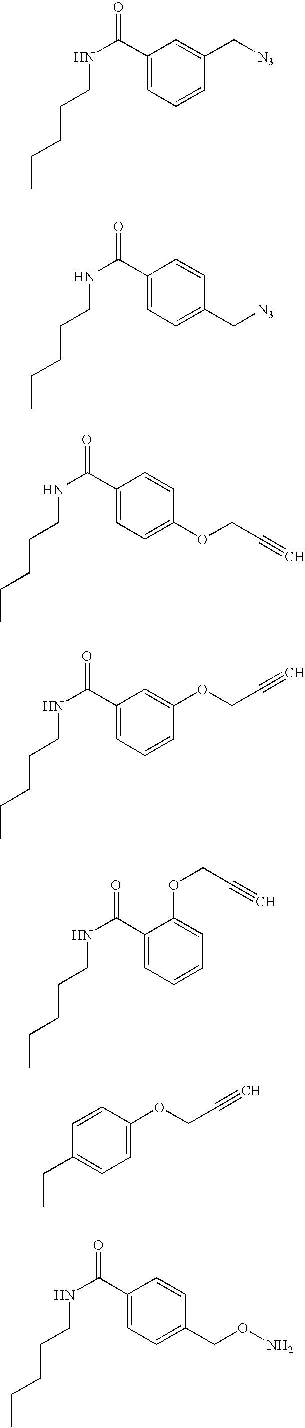

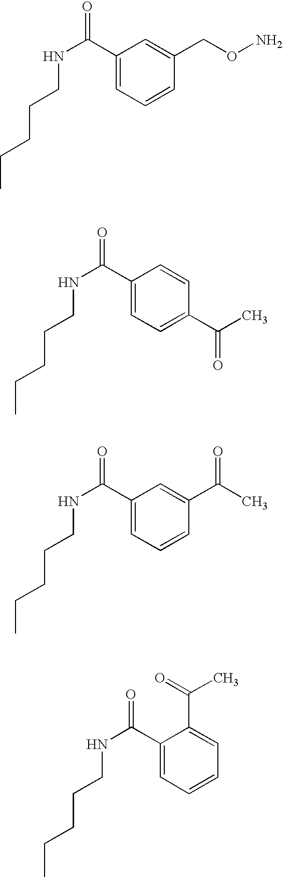

The present invention also provides a method for dimerizing antibody fragments which can be used for production of bi-specific constructs, reducing or eliminating mispairing between different antibody fragments.

As mentioned above, the present invention provides a process for dimerization of two antibody fragments of antibodies comprising the steps of

(a) introducing a first chemical group to the C-terminus of the first antibody fragment,

(b) introducing a second chemical group to the C-terminus of the second antibody fragment, and

(c) reacting the first chemical group with the second chemical group to form a covalent linkage of the two antibody fragments.

In another aspect the present invention provides a process for dimerization of two antibody fragments of antibodies, comprising the steps of

(a′) introducing a first chemical group to the C-terminus of the first antibody fragment by reaction with an enzyme in the presence of a nucleophile,

(b′) introducing a second chemical group to the C-terminus of the second antibody fragment by reaction with an enzyme in the presence of a nucleophile, and

(c) reacting the first chemical group with the second chemical group to form a covalent linkage of the two antibody fragments.

In another aspect the present invention provides a process for dimerization of two antibody fragments of antibodies, comprising the steps of

(a′) introducing a first chemical group to the C-terminus of the first antibody fragment by reaction with an enzyme in the presence of a nucleophile,

(b″) introducing a third chemical group to the C-terminus of the second antibody fragment by reaction with an enzyme in the presence of a nucleophile,

(b′″) reacting the third chemical group with a molecule bearing a fourth and a second chemical group to attach said molecule covalently to the C-terminus of the second anti-body fragment by reaction of the third chemical group and the fourth chemical group, and

(c) reacting the first chemical group with the second chemical group to form a covalent linkage of the two antibody fragments.

In one embodiment, in any of the aspects or embodiments above, step (a) comprises modifying the C-terminal residue of a HC polypeptide of a first antibody fragment to comprise a first chemical group, and step (b) comprises modifying the C-terminal residue of a HC polypeptide of a second antibody fragment to comprise a second (or third) chemical group.

In yet another embodiment, the HC polypeptides comprise at least two, or all three complementarity-determining regions (CDR) of an antibody.

In one embodiment of the invention, said first chemical group and said second chemical group are different from each other.

In another embodiment of the invention, said first chemical group and said second chemical group are independently selected from the group consisting of alkyne, azide, O-alkylated hydroxylamine, ketone, aldehyde, hydrazone and O-acylated hydroxylamine.

In another embodiment of the invention, a reaction between an azide and an alkyne is used to form the linkage between the two antibody fragments, so that the first chemical group is an azide, and the second an alkyne, or vice versa. In yet another embodiment of the invention said reaction between an azide and an alkyne is catalyzed by copper(I)-ions.

In another embodiment of the invention, a reaction between an O-alkylated hydroxylamine and a ketone or an aldehyde is used to form the linkage between the two antibody fragments, so that the first chemical group is an O-alkylated hydroxylamine, and the second chemical group is a ketone or an aldehyde, or vice versa.

In one aspect of the invention, each reacting group of such a pair is introduced to the C-terminus of a HC-polypeptide by an enzyme-catalyzed reaction. In one embodiment, at least one of the reacting groups is an hydroxylamine or an azide.

The enzyme catalyzed modification of the C-termini of the antibody fragments may be performed by a variety of enzymes, including, but not limited to serine proteases such as serine carboxypeptidases. In one embodiment, the enzyme is carboxypeptidase Y (CPY). In another embodiment the enzyme is a variant or a fragment of carboxypeptidase Y, which variant or fragment retains the ability to catalyse a reaction, by which the C-terminal amino acid of a polypeptide is replaced by a different chemical moiety. Several variants of carboxypeptidase Y are known in the art; see e.g. WO 98/38285.

In another embodiment of the invention, said nucleophile is selected from the group consisting of

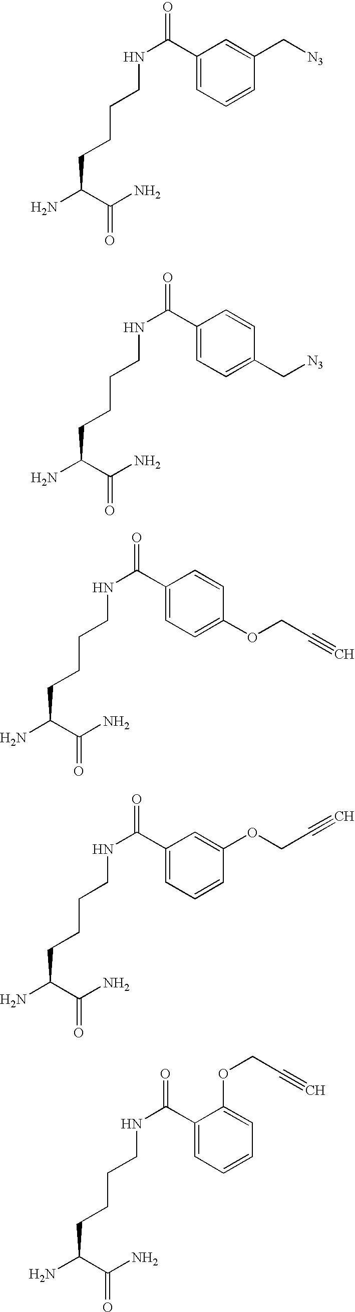

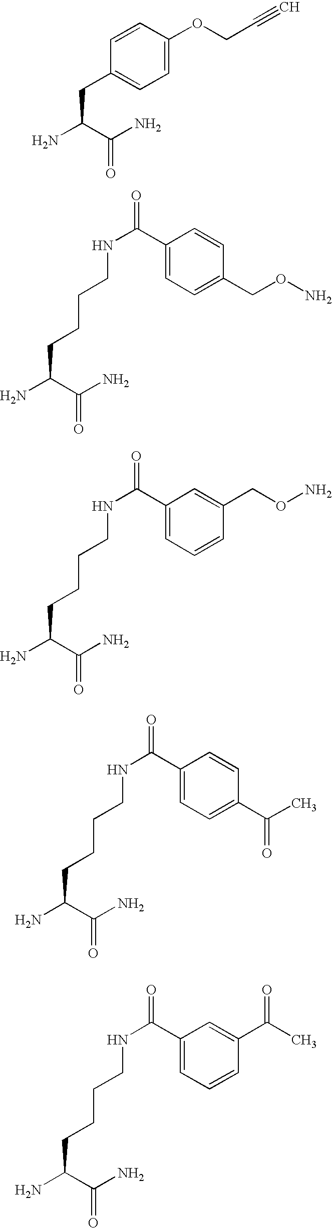

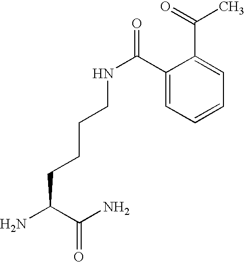

In another embodiment of the invention, said reaction in step (c) forms an 1,2,3-triazole.

In another embodiment of the invention, said reaction in step (c) forms an oxime or a hydrazone.

In one embodiment of the methods of invention, the C-terminal residues of the anti-body fragments to be coupled are Ala residues, preferably Leu-Leu-Ala peptide sequences. In another embodiment, an Ala residue is added to the C-terminus of each antibody fragment prior to introducing a first chemical group and a second chemical group to the C-terminus of each respective antibody fragment, by pre- or post-translational elongation. In another embodiment, a Leu-Leu-Ala polypeptide is added to the C-terminus of each antibody fragment prior to introducing a first chemical group and a second chemical group to the C-terminus of each respective antibody fragment, by pre- or post translational elongation.

In another aspect, the present invention relates to an antibody-fragment such as a Fab-fragment wherein the C-terminal amino acid sequence is Leu-Leu-Ala.

### Antibody Fragments

Antibodies (or “immunoglobulins”) are proteins secreted by mammalian (e.g., human) B lymphocyte-derived plasma cells in response to the appearance of an antigen. Though multimers can form, the basic unit of each antibody is a “Y”-shaped molecule that consists of two identical heavy chains and two identical light chains.

Specifically, each such antibody contains a pair of identical heavy chains (HCs) and a pair of identical light chains (LCs). Each LC has one variable domain (VL) and one constant domain (CL), while each HC has one variable (VH) and three constant domains (CH1, CH2, and CH3). Each variable domain, in turn, comprises three complementarity-determining regions (CDRs) interspersed by framework regions (FRs). The CH1 and CH2 domains are connected by a hinge region. Each polypeptide is characterized by a number of intrachain disulphide bridges and polypeptides are interconnected by additional disulphide bridges. In addition to disulphide bridging the polypeptides, the polypeptide chains also are associated due to ionic interactions (which interactions are directly relevant to many aspects of the invention described herein).

There are five types of heavy chain: γ, δ, α, μ and ε (or G, D, A, M, and E). They define classes of immunoglobulins. H chains of all isotypes associate with light (L) chains of two isotypes—k and I. Thus, the basic H2L2 composition of an antibody can be specified in terms of its H and L isotypes; e.g., e2k2, (m2I2)5, etc. Based on the differences in their heavy chains, immunoglobulin molecules are divided into five major classes: IgG, IgM, IgA, IgE, and IgD. Immunoglobulin G (“IgG”) is the predominant immunoglobulin of internal components such as blood, cerebrospinal fluid and peritoneal fluid (fluid present in the abdominal cavity). IgG is the only class of immunoglobulin that crosses the placenta, conferring the mother's immunity on the fetus. IgG makes up 80% of the total immunoglobulins. It is the smallest immunoglobulin, with a molecular weight of 150,000 Daltons. Thus it can readily diffuse out of the body's circulation into the tissues. All currently approved antibody drugs comprise IgG or IgG-derived molecules.

In some species, the immunoglobulin classes are further differentiated according to subclasses, adding another layer of complexity to antibody structure. In humans, for example, IgG antibodies comprise four IgG subclasses—IgG1, IgG2, IgG3, and IgG4. Each subclass corresponds to a different heavy chain isotype, designated g1 (IgG1), g2 (IgG2), g3 (IgG3), g4 (IgG4), al (IgA1) or a2 (IgA2).

In mammals (and certain other chordates), the reaction between antibodies and an antigen (which is usually associated with an infectious agent) leads to elimination of the antigen and its source. This reaction is highly specific, that is, a particular antibody usually reacts with only one type of antigen. The antibody molecules do not destroy the infectious agent directly, but, rather, “tag” the agent for destruction by other components of the immune system. In mammals such as humans, the tag is constituted by the CH2-CH3 part of the antibody, commonly referred to as the Fc domain.

Immunoglobulins can be converted into smaller fragments that still retain the antigen binding site and consequently the specificity towards an antigen. One such antigen-binding fragments have been designated Fab (antigen binding fragment). A Fab consists of two polypeptides, one containing the light chain variable and constant domains VL-CL, the other a truncated heavy chain containing the variable domain and one constant domain VH-CH1. If the hinge region is also included disulfide bridge formation can occur between two Fab fragments giving Fab2 fragments. Thus, the Fc domain is absent in Fab and Fab2 fragments. Just as in intact IgG immunoglobulins, the light and heavy chain are linked together by a disulfide bond.

The term “antibody fragment” as used herein means an antigen-binding fragment of an antibody, the antigen-binding fragment comprising a HC polypeptide comprising at least a portion of a full-length HC. Typically, the antigen-binding fragment comprises only one HC polypeptide. The HC polypeptide may comprise, e.g., one, two or all three CDRs of the VH of an antibody. The antibody fragment can further comprise an LC polypeptide comprising at least a portion of a full-length LC. The LC polypeptide may comprise, e.g., one, two or, all three CDRs of the VL of an antibody. Examples of antibody fragments include Fab (also termed “FAB” herein), Fab′, Fv (typically the VL and VH domains of a single arm of an antibody), single-chain Fv (scFv), Fd fragments (typically the VH and CH1 domain), and dAb (typically a VH domain) fragments; VH, VhH, and V-NAR domains; as well as a monovalent versions of a full-length antibody (comprising a full-length HC and a full-length LC); monovalent versions of minibodies, diabodies, triabodies, tetrabodies, and kappa bodies (see, e.g., Ill et al., Protein Eng 1997; 10: 949-57); monovalent versions of camel IgG; monovalent versions of IgNAR; and one or more isolated VH CDRs or a functional paratope, where isolated CDRs or antigen-binding residues or polypeptides can be associated or linked together so as to form a functional antibody fragment. Various types of antibody fragments suitable for dimerization have been described or reviewed in, e.g., Holliger and Hudson, Nat Biotechnol 2005; 23, 1126-1136; WO2005040219, and published U.S. Patent Applications 20050238646 and 20020161201.

The antibody fragments may comprise natural amino acids encoded by the genetic code, natural amino acids not encoded by the genetic code, as well as synthetic amino acids. Natural amino acids which are not encoded by the genetic code are e.g. hydroxyproline, γ-carboxy-glutamic acid, ornithine, phophoserine, D-alanine, D-glutamic acid. Synthetic amino acids comprise amino acids manufactured by organic synthesis, e.g. D-isomers of the amino acids encoded by the genetic code and Aib (α-aminoisobutyric acid), Abu (α-aminobutyric acid), Tle (tert-butylglycine), and β-alanine.

In one aspect of the invention the C-terminal amino acid of at least one of the two antibody fragments or Fab-fragments has a non polar-side chain. In one embodiment, the C-terminal amino acid of at least one of the two antibody fragments or Fab-fragments is -Ala. In another embodiment of the invention the C-terminal amino acid sequence of at least one of the two antibody fragments or Fab-fragments is Leu-Leu-Ala. This sequence has been shown to advantageous for a enzyme reaction comprising an enzyme such as CPY. In one embodiment, the Ala residue or Leu-Leu-Ala peptide sequence are introduced at the C-terminal of at least one of the antibody fragments before coupling.

In one embodiment, each HC polypeptide of the antibody fragments comprises all three CDRs from an antibody. In another embodiment, each HC polypeptide of the antibody fragments comprises all three CDRs from an antibody HC, and is associated with an LC polypeptide comprising 1, 2, or 3 CDRs from an antibody LC. In another embodiment, each HC polypeptide of the antibody fragments comprises all three CDRs from an antibody HC, and is associated with an LC polypeptide comprising all three CDRs from an antibody LC.

The process according to the present invention may provide bispecific dimers of antibody fragments (e.g., bispecific Fab2 fragments). Thus, in one embodiment of the invention the two antibody fragments are different from each other, binding different antigens or different epitopes on the same antigen.

In another aspect, the present invention relates to an antibody-fragment such as a Fab-fragment wherein the C-terminal amino acid sequence is Leu-Leu-Ala.

The following describes four exemplary processes for producing dimerized antibody fragments according to the present invention. Though exemplified for dimerization of Fab (also termed “FAB”) fragments below, the same process can be applied to other antibody fragments.

### Process A

Step 1: Preparation of the First FAB-Fragment Bearing a First Chemical Group at its C-Terminus

A first FAB-fragment with a suitable C-terminal amino acid sequence such as e.g. -LLA, is incubated together with carboxypeptidase Y (CPY) in the presence of a nucleophile, which is bearing a moiety R1^{cg} with a first chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R1^{c} and R1^{c-1} are the amino acid residues at the positions at the positions of the first FAB-fragment. FAB² is a radical of the second FAB-fragment.

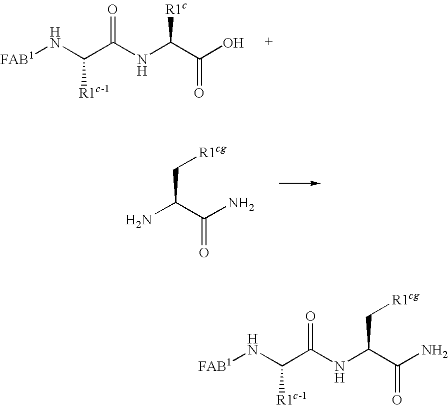

Step 2: Preparation of the Second FAB-Fragment Bearing a Second Chemical Group at its C-Terminus

A second FAB-fragment with a suitable C-terminal amino acid sequence such as e.g. -LLA, is incubated together with a suitable enzyme such as, e.g., carboxypeptidase Y (CPY) in the presence of a nucleophile, which is bearing a moiety R2^{cg} with a second chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R2^{c} and R2^{c-1} are the amino acid residues at the positions at the positions of the second FAB-fragment. FAB² is a radical of the second FAB-fragment.

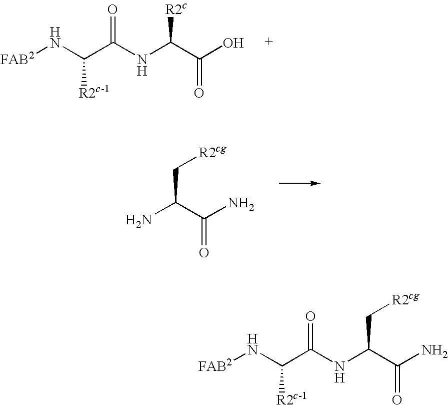

Step 3: Reacting the First and the Second Chemical Group to Link the First and the Second FAB-Fragment

The moiety with first chemical group, which is attached to the first transpeptidated FAB-fragment and which is not accessible or present in the second FAB-fragment, may be reacted with the moiety with second chemical group, which is attached to the second transpeptidated FAB-fragment and which is accessible or not present in the first transpeptidated FAB-fragment to form a linking moiety R1Link2. Examples for pairs of chemical groups which may reacted with each other could be e.g.: alkynes and azides, which may react under suitable conditions to triazole compounds, such as e.g. copper(I) catalysis, or ketones or aldehydes and O-alkylated hydroxylamines, which may react at a suitable pH to oximes.

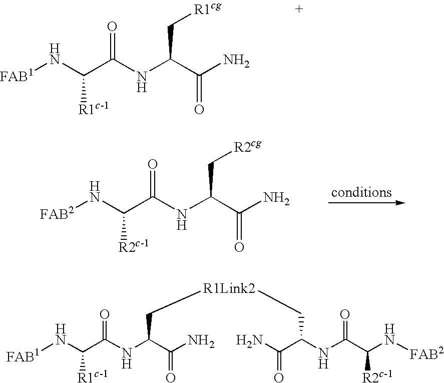

### Process B

Step 1: Preparation of the First FAB-Fragment Bearing a First Chemical Group at its C-Terminus

A first FAB-fragment with a suitable C-terminal amino acid sequence such as e.g. -LLA, is incubated together with carboxypeptidase Y (CPY) in the presence of a nucleophile, which is bearing a moiety with a first chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R1^{c} and R1^{c-1} are the amino acid residues at the positions at the positions of the first FAB-fragment.

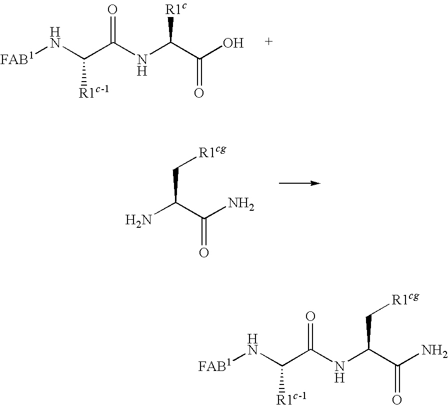

Step 2. Preparation of the Second FAB-Fragment Bearing a Third Chemical Group at its C-Terminus.

A second FAB-fragment with a suitable C-terminal amino acid sequence such as e.g. -LLA, is incubated together with carboxypeptidase Y (CPY) in the presence of a nucleophile, which is bearing a moiety with a moiety R3^{cg} comprising a third chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R2^{c} and R2^{c-1} are the amino acid residues at the positions at the positions of the second FAB-fragment. FAB² is a radical of the second FAB-fragment. FAB² is a radical of the second FAB-fragment.

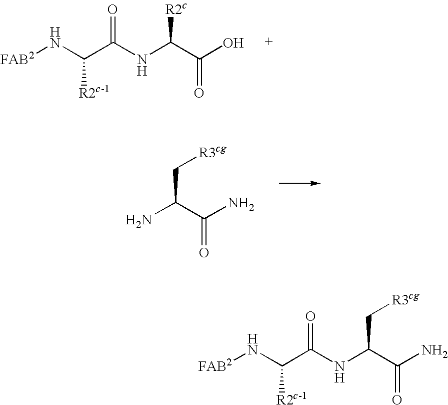

Step 3. Reaction with a Molecule which is Bearing a Second and a Fourth Chemical Group.

The second FAB-fragment which comprises a third chemical group obtained in the preceding step may be reacted with a molecule, which diradical is Mol, having a moiety R4^{cg} comprising a fourth chemical group and a moiety R2^{cg} comprising a second chemical group. Examples for such chemical groups could be e.g. alkynes, azides, ketones, aldehydes, O-alkylated hydroxylamines, hydrazines. A transpeptidated reaction product may be formed. A covalent linkage may be formed by reaction of the third chemical group with the fourth chemical group forming a linking moiety R3linkage4.

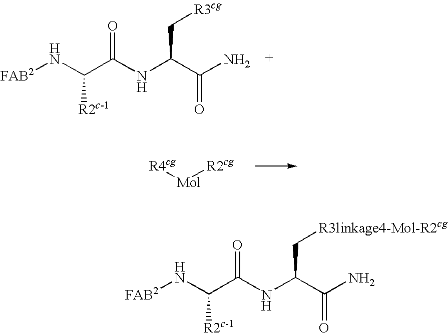

Step 4. Linkage of the two FAB-Fragments

The FAB-fragments obtained in Step 1 and Step 3 respectively, may form a dimerized compound by reaction of the moiety with first chemical group, which is attached to the first transpeptidated FAB-fragment and which is not accessible or present in the second FAB-fragment, obtained in step 3, may be reacted with the moiety with second chemical group, which is attached to the second transpeptidated FAB-fragment and which is accessible or not present in the first transpeptidated FAB-fragment to form a linking moiety R1Lnk2. Examples for pairs of chemical groups which may reacted with each other could be e.g.: alkynes and azides, which may react under suitable conditions to triazole compounds, such as e.g. copper(I) catalysis, or ketones or aldehydes and O-alkylated hydroxylamines, which may react at a suitable pH to oximes.

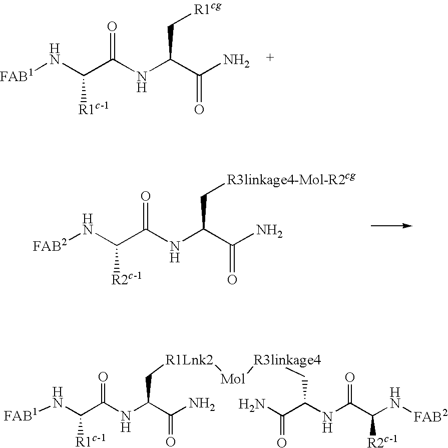

### Process C

### Step 1: Preparation of the First FAB-Fragment Bearing a First Chemical Group at its C-Terminus

A first FAB-fragment with a suitable C-terminal amino acid sequence such as e.g. -LLA, is incubated together with carboxypeptidase Y (CPY) in the presence of a nucleophile, which is bearing a moiety R1^{cg} with a first chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R^{1c} and R1^{c-1} are the amino acid residues at the positions at the positions of the first FAB-fragment. FAB² is a radical of the second FAB-fragment.

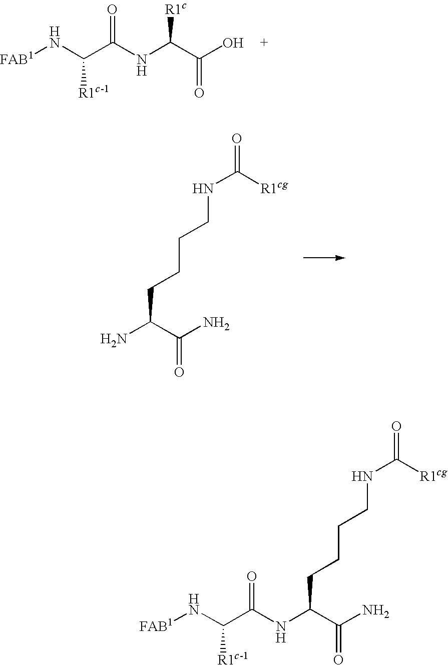

### Step 2: Preparation of the Second FAB-Fragment Bearing a Second Chemical Group at its C-Terminus

A second FAB-fragment with a suitable C-terminal amino acid sequence such as e.g. -LLA, is incubated together with a suitable enzyme such as, e.g., carboxypeptidase Y (CPY) in the presence of a nucleophile, which is bearing a moiety R2^{cg} with a second chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R^{2c} and R2^{c-1} are the amino acid residues at the positions at the positions of the second FAB-fragment. FAB² is a radical of the second FAB-fragment.

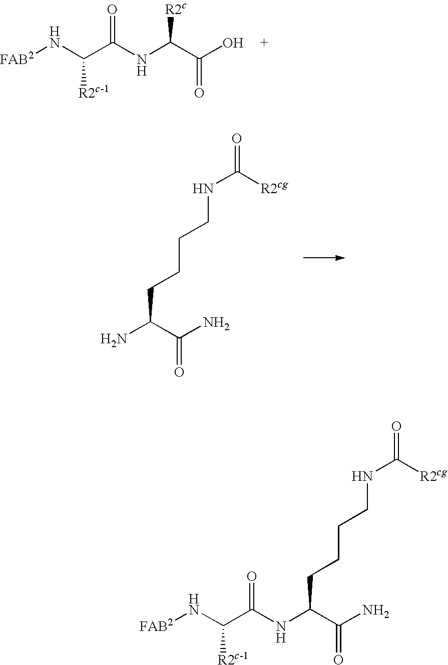

Step 3: Reacting the First and the Second Chemical Group to Link the First and the Second FAB-Fragment

The moiety with first chemical group, which is attached to the first transpeptidated FAB-fragment and which is not accessible or present in the second FAB-fragment, may be reacted with the moiety with second chemical group, which is attached to the second transpeptidated FAB-fragment and which is accessible or not present in the first transpeptidated FAB-fragment to form a linking moiety R1Link2. Examples for pairs of chemical groups which may reacted with each other could be e.g.: alkynes and azides, which may react under suitable conditions to triazole compounds, such as e.g. copper(I) catalysis, or ketones or aldehydes and O-alkylated hydroxylamines, which may react at a suitable pH to oximes.

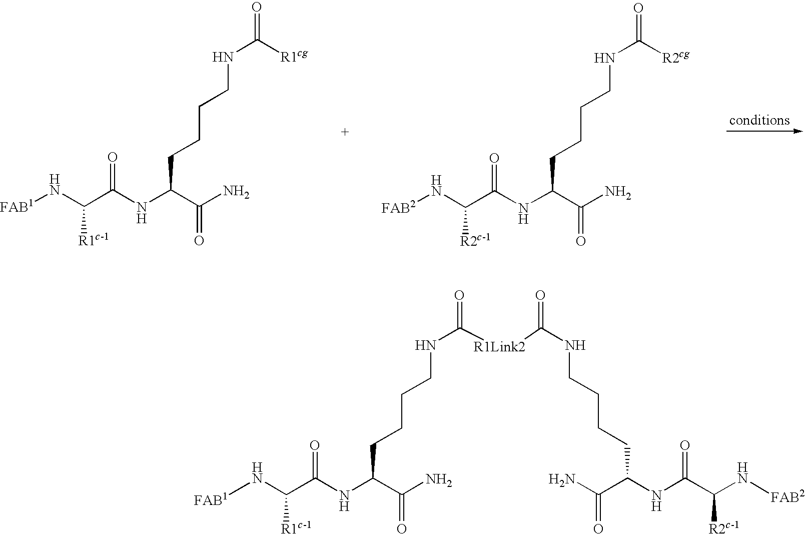

### Process D

Step 1: Preparation of the First FAB-Fragment Bearing a First Chemical Group at its C-Terminus

A first FAB-fragment with a suitable C-terminal amino acid sequence such as e.g. -LLA, is incubated together with carboxypeptidase Y (CPY) in the presence of a nucleophile, which is bearing a moiety with a first chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R1^{c} and R1^{c-1} are the amino acid residues at the positions at the positions of the first FAB-fragment.

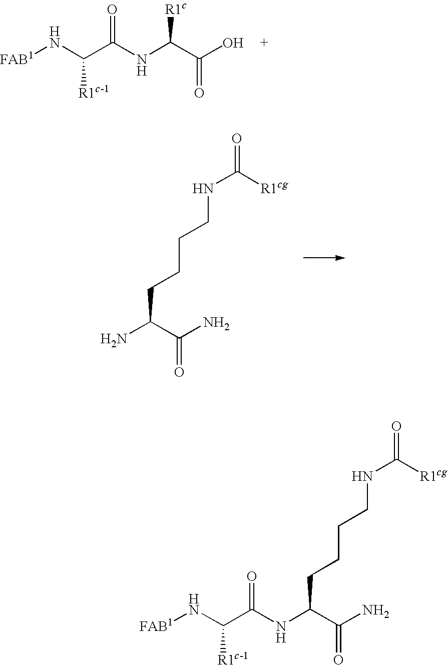

Step 2. Preparation of the Second FAB-Fragment Bearing a Third Chemical Group at its C-Terminus.

A second FAB-fragment with a suitable C-terminal amino acid sequence such as e.g. -LLA, is incubated together with carboxypeptidase Y (CPY) in the presence of a nucleophile, which is bearing a moiety with a moiety R3^{cg} comprising a third chemical group, which is not present in the second FAB-fragment. Examples for such chemical groups could be e.g. alkynes, azides, ketones, aldehydes, O-alkylated hydroxylamines, hydrazines. A transpeptidated reaction product may be formed. R2^{c} and R2^{c-1} are the amino acid residues at the positions at the positions of the second FAB-fragment. FAB² is a radical of the second FAB-fragment. FAB² is a radical of the second FAB-fragment.

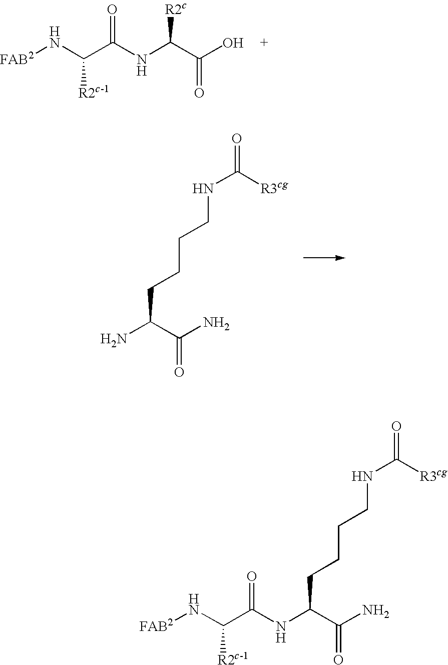

Step 3. Reaction with a Molecule which is Bearing a Second and a Fourth Chemical Group.

The second FAB-fragment which comprises a third chemical group obtained in the preceding step may be reacted with a molecule, which diradical is Mol, having a moiety R4^{cg} comprising a fourth chemical group and a moiety R2^{cg} comprising a second chemical group. Examples for such chemical groups could be e.g. alkynes, azides, ketones, aldehydes, O-alkylated hydroxylamines, hydrazines. A transpeptidated reaction product may be formed. A covalent linkage may be formed by reaction of the third chemical group with the fourth chemical group forming a linking moiety R3linkage4.

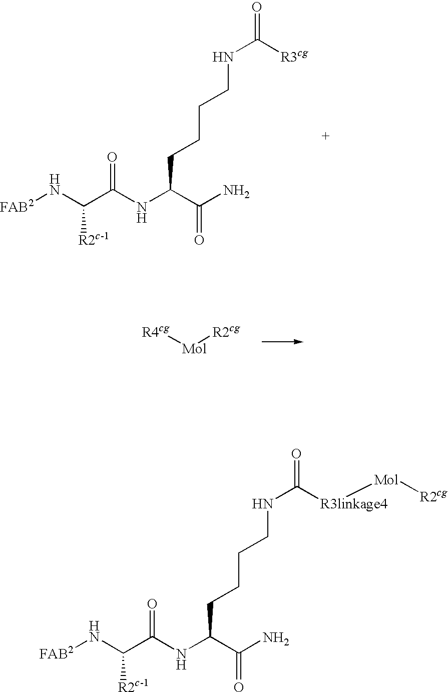

Step 4. Linkage of the two FAB-Fragments

The FAB-fragments obtained in Step 1 and Step 3 respectively, may form a dimerized compound by reaction of the moiety with first chemical group, which is attached to the first transpeptidated FAB-fragment and which is not accessible or present in the second FAB-fragment, obtained in step 3, may be reacted with the moiety with second chemical group, which is attached to the second transpeptidated FAB-fragment and which is accessible or not present in the first transpeptidated FAB-fragment to form a linking moiety R1Lnk2. Examples for pairs of chemical groups which may reacted with each other could be e.g.: alkynes and azides, which may react under suitable conditions to triazole compounds, such as e.g. copper(I) catalysis, or ketones or aldehydes and O-alkylated hydroxylamines, which may react at a suitable pH to oximes.

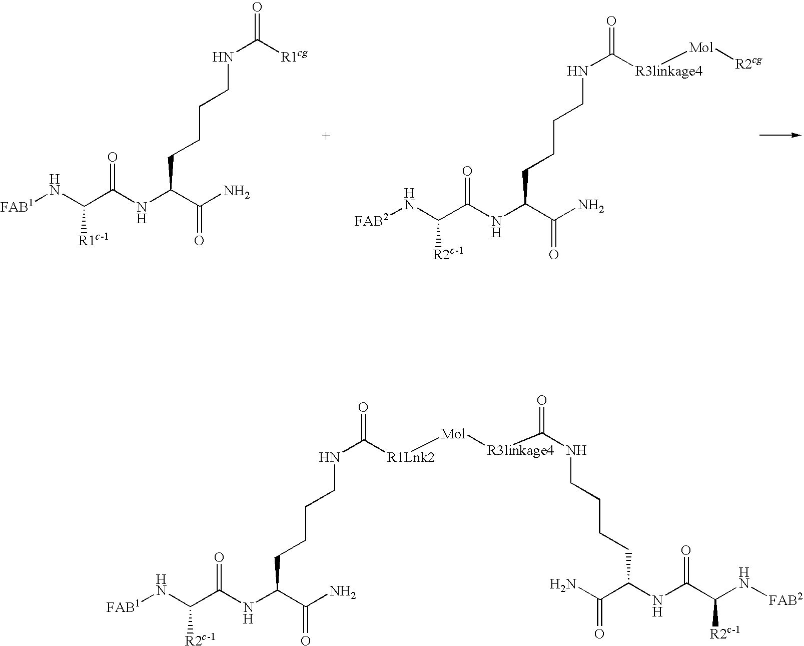

### Conjugation

The dimerized antibody fragments according to the present invention may also or alternatively be conjugated, i.e. attachment (conjugation) of a chemical group, e.g. a non-polypeptide moiety.

Hence, in one embodiment, the process further comprises the simultaneous and/or subsequent step of conjugating at least one of the constituent antibody polypeptides with a chemical group. Such conjugation may be performed via a reduced cysteine residue, or it may be performed via a glutamic acid residue.

It is to be understood that conjugation may be conducted on one of the constituent antibody fragments before synthesis of the dimerized antibody fragments, or it may be conducted after the dimerized antibody fragment has been synthesized.

In one embodiment, the chemical group is a protractor group, i.e. a group which upon conjugation to a polypeptide increases the circulation half-life of said polypeptide, when compared to the un-modified polypeptide. The specific principle behind the protractive effect may be caused by increased size, shielding of peptide sequences that can be recognized by peptidases or antibodies, or masking of glycanes in such way that they are not recognized by glycan specific receptors present in e.g. the liver or on macrophages, preventing or decreasing clearance. The protractive effect of the protractor group can e.g. also be caused by binding to blood components such as albumin, or unspecific adhesion to vascular tissue. The conjugated polypeptide should substantially preserve its biological activity.

In one embodiment, only one of the antibody fragments is conjugated to a chemical group such as, e.g. a non-polypeptide moiety.

In one embodiment of the invention the protractor group is selected from the group consisting of:

(a) A low molecular organic charged radical (15-1,000 Da), which may contain one or more carboxylic acids, amines sulfonic acids, phosphonic acids, or combination thereof.

(b) A low molecular (15-1,000 Da) neutral hydrophilic molecule, such as cyclodextrin, or a polyethylene chain which may optionally branched.

(c) A low molecular (15-1,000 Da) hydrophobic molecule such as a fatty acid or cholic acid or derivatives thereof.

(d) Polyethyleneglycol with an average molecular weight of 2,000-60,000 Da.

(e) A well defined precision polymer such as a dendrimer with an exact molecular mass ranging from 700 to 20,000 Da, or more preferable between 700-10,000 Da.

(f) A substantially non-immunogenic polypeptide such as albumin, an antibody or a part thereof, e.g. an albumin fragment or an antibody fragment optionally containing an Fc-domain.

(g) A high molecular weight organic polymer such as dextran.

In another embodiment of the invention the protractor group is selected from the group consisting of dendrimers, polyalkylene oxide (PAO), including polyalkylene glycol (PAG), such as polyethylene glycol (PEG) and polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, and dextran, including carboxymethyldextran. In one particularly interesting embodiment of the invention, the protractor group is a PEG group.

The term “branched polymer”, or interchangebly “dendritic polymer”, “dendrimer” or “dendritic structure” means an organic polymer assembled from a selection of monomer building blocks of which, some contains branches.

In one embodiment of the invention the protractor group is a selected from the group consisting of serum protein binding-ligands, such as compounds which bind to albumin, like fatty acids, C5-C24 fatty acid, aliphatic diacid (e.g. C5-C24). Other examples of protractor groups includes small organic molecules containing moieties that under physiological conditions alters charge properties, such as carboxylic acids or amines, or neutral substituents that prevent glycan specific recognition such as smaller alkyl substituents (e.g., C1-C5 alkyl). In one embodiment of the invention the protractor group is albumin.

In one embodiment, the chemical group is a non-polypeptide.

In one interesting embodiment, the chemical group is a polyethyleneglycol (PEG), in particular one having an average molecular weight of in the range of 500-100,000, such as 1,000-75,000, or 2,000-60,000.

Conjugation can be conducted as disclosed in WO 02/077218 A1 and WO 01/58935 A2.

Particularly interesting is the use of PEG as a chemical group for conjugation with the protein. The term “polyethylene glycol” or “PEG” means a polyethylene glycol compound or a derivative thereof, with or without coupling agents, coupling or activating moeities (e.g., with thiol, triflate, tresylate, azirdine, oxirane, pyridyldithio, vinyl sulfone, or preferably with a maleimide moiety). Compounds such as maleimido monomethoxy PEG are exemplary of activated PEG compounds of the invention.

PEG is a suitable polymer molecule, since it has only few reactive groups capable of cross-linking compared to polysaccharides such as dextran. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting anti-body fragment conjugates are more homogeneous and the reaction of the polymer molecules with the antibody fragment is easier to control.

To effect covalent attachment of the polymer molecule(s) to the antibody fragment, the hydroxyl end groups of the polymer molecule are provided in activated form, i.e. with reactive functional groups. Suitable activated polymer molecules are commercially available, e.g. from Shearwater Corp., Huntsville, Ala., USA, or from PolyMASC Pharmaceuticals plc, UK. Alternatively, the polymer molecules can be activated by conventional methods known in the art, e.g. as disclosed in WO 90/13540. Specific examples of activated linear or branched polymer molecules for use in the present invention are described in the Shearwater Corp. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPDX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the antibody fragment (examples of which are given further above), as well as the functional groups of the polymer (e.g. being amine, hydroxyl, carboxyl, aldehyde, sulfydryl, succinimidyl, maleimide, vinysulfone or haloacetate). The PEGylation may be directed towards conjugation to all available attachment groups on the antibody fragment or may be directed towards one or more specific attachment groups, e.g. the N-terminal amino group. Furthermore, the conjugation may be achieved in one step or in a stepwise manner (e.g. as described in WO 99/55377).

It will be understood that the PEGylation is designed so as to produce the optimal molecule with respect to the number of PEG molecules attached, the size and form of such molecules (e.g. whether they are linear or branched), and where in the antibody fragment such molecules are attached. The molecular weight of the polymer to be used will be chosen taking into consideration the desired effect to be achieved. For instance, if the primary purpose of the conjugation is to achieve a conjugate having a high molecular weight and larger size (e.g. to reduce renal clearance), one may choose to conjugate either one or a few high molecular weight polymer molecules or a number of polymer molecules with a smaller molecular weight to obtain the desired effect. Preferably, however, several polymer molecules with a lower molecular weight will be used. This is also the case if a high degree of epitope shielding is desired. In such cases, 2-8 polymers with a molecular weight of e.g. about 5,000 Da, such as 3-6 such polymers, may for example be used. As the examples below illustrate, it may be advantageous to have a larger number of polymer molecules with a lower molecular weight (e.g. 4-6 with a M_{w} of 5,000) compared to a smaller number of polymer molecules with a higher molecular weight (e.g. 1-3 with a MW of 12,000-20,000) in terms of improving the functional in vivo half-life of the conjugate, even where the total molecular weight of the attached polymer molecules in the two cases is the same or similar. It is believed that the presence of a larger number of smaller polymer molecules provides the antibody fragment with a larger diameter or apparent size than e.g. a single yet larger polymer molecule, at least when the polymer molecules are relatively uniformly distributed on the antibody fragment surface.

It has further been found that advantageous results are obtained when the apparent size (also referred to as the “apparent molecular weight” or “apparent mass”) of at least a major portion of the conjugate of the invention is at least about 50 kDa, such as at least about 55 kDa, such as at least about 60 kDa, e.g. at least about 66 kDa. This is believed to be due to the fact that renal clearance is substantially eliminated for conjugates having a sufficiently large apparent size. In the present context, the “apparent size” of an antibody fragment can be determined by the SDS-PAGE method.

Furthermore, excessive polymer conjugation can lead to a loss of activity (binding affinity) of the antibody fragment to which the chemical group (e.g. a non-polypeptide moiety) is conjugated (see further below). This problem can be eliminated, e.g., by removal of attachment groups located in the CDRs or variable regions, or by reversible blocking the functional site prior to conjugation so that the binding sites of the antibody fragment is blocked during conjugation. Specifically, the conjugation between the antibody fragment and the chemical group (e.g. non-polypeptide moiety) may be conducted under conditions where the binding site of the antibody fragment is blocked by a helper molecule. Preferably, the helper molecule is one, which specifically binds to the antibody fragment.

The antibody fragment is preferably to interact with the helper molecule before effecting conjugation. Often it is advantageous to use the antigen or an antigen-mimic as helper molecule. This ensures that the binding site of the antigen fragment is shielded or protected and consequently unavailable for derivatization by the chemical group (e.g. non-polypeptide moiety) such, as a polymer.

Following its elution from the helper molecule, the conjugate of the chemical group and the antibody fragment can be recovered with at least a partially preserved binding site.

### Pharmaceutical Compositions

The antibody fragment dimers according to the present invention are applicable as pharmaceutical compositions for the treatment of disorders or diseases in patients.

In another aspect, the present invention includes within its scope pharmaceutical compositions comprising an antibody fragment dimer as an active ingredient, or a pharmaceutically acceptable salt thereof together with a pharmaceutically acceptable carrier or diluent.

The compounds of the invention may be formulated into pharmaceutical compositions comprising the compounds and a pharmaceutically acceptable carrier or diluent. Such carriers include water, physiological saline, ethanol, polyols, e.g., glycerol or propylene glycol, or vegetable oils. As used herein, “pharmaceutically acceptable carriers” also encompasses any and all solvents, dispersion media, coatings, antifungal agents, preservatives, isotonic agents and the like. Except insofar as any conventional medium is incompatible with the active ingredient and its intended use, its use in the compositions of the present invention is contemplated.

The compositions may be prepared by conventional techniques and appear in conventional forms, for example, capsules, tablets, solutions or suspensions. The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilised and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain an antibody fragment dimer dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with a suitable buffer.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

The antibody fragment dimer of the invention may be administered to a mammal, especially a human in need of such treatment, prevention, elimination, alleviation or amelioration of various diseases or disorders. Such mammals also include animals, both domestic animals, e.g. household pets, and non-domestic animals such as wildlife.

Usually, dosage forms suitable for intravenous or subcutaneous administration comprise from about 0.001 mg to about 100 mg, preferably from about 0.01 mg to about 50 mg of the antibody fragment dimer admixed with a pharmaceutically acceptable carrier or diluent.

The antibody fragment dimer may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

The present invention is further illustrated by the following examples which, however, are not to be construed as limiting the scope of protection. The features disclosed in the foregoing description and in the following examples may, both separately or in any combination thereof, be material for realising the invention in diverse forms thereof.

### EXAMPLES

### Example 1

### Dimerization of a IL-20 FAB-Fragment

### Step 1: Transpeptidation Reaction with (S)-2-Amino-6-(3-(azidomethyl)benzoylamino)hexanoic amide

The buffer was changed of a 0.51 mg/ml solution (1.37 ml, 14 nmol) of an IL-20 FAB-fragment which at its C-terminus was elongated with leucylleucylalanine in a buffer consisting of 30 mM sodium phosphate buffer and 150 mM sodium chloride and a pH of 7.2 to a buffer (0.040 ml) consisting of 0.25 mM HEPES and 5 mM EDTA with a pH of 8.0 by centrifugation in a Biomax centrifuge vial with a cut off of 10 000 Da. A solution of (S)-2-amino-6-(3-(azidomethyl)benzoylamino)hexanoic amide (7.2 mg, 16800 nmol) in a buffer consisting of 0.25 mM HEPES and 5 mM EDTA with a pH of 8.0 (0.020 ml) was prepared. The pH of this solution was adjusted to pH 8 by addition of a 4 M aqueous solution of sodium hydroxide (0.003 ml). A part of this solution (0.005 ml, 4200 nmol) was added to the solution of the FAB-fragment. The pH was found to be 7.97. A solution of CPY in water (200 U/ml, 0.008 ml). The reaction mixture was gently shaken at 30° C. for 24 h. A freshly prepared 10 mM solution of phenylmethylsulfonyl fluoride in dry isopropanol (0.0002 ml) was added. The reaction mixture was shaken gently for 30 min at room temperature. Another portion of the 10 mM pheylmethylsulfonyl fluoride solution (0.0053 ml) was added. The reaction mixture was concentrated by centrifugation in a Biomax centrifuge vial with a cut off of 10 000 Da. It was diluted with a 2% solution of 2,6-lutidine in water (0.5 ml). A freshly prepared 100 mM solution of phenylmethylsulfonyl fluoride in dry isopropanol (0.0045 ml) was added. The reaction mixture was concentrated by centrifugation in a Biomax centrifuge vial with a cut off of 10 000 Da to a volume of 0.100 ml. A NAP-5 column was equilibrated with a 2% solution of 2,6-lutidine in water. The solution of the reaction mixture was applied to the column. The protein was washed out with a 2% solution of 2,6-lutidine in water. The solution containing the protein was concentrated by centrifugation in a Biomax centrifuge vial with a cut off of 10 000 Da to a volume of 0.040 ml.

### Step 2: Transpeptidation Reaction with (2S)-2-Amino-3-(4-(prop-2-ynyloxy)phenyl)propion-amide

The buffer was changed of a 0.51 mg/ml solution (1.37 ml, 14 nmol) of an IL-20 FAB-fragment which at its C-terminus was elongated with leucylleucylalanine in a buffer consisting of 30 mM sodium phosphate buffer and 150 mM sodium chloride and a pH of 7.2 to a buffer (0.040 ml) consisting of 0.25 mM HEPES and 5 mM EDTA with a pH of 8.0 by centrifugation in a Biomax centrifuge vial with a cut off of 10 000 Da. A solution of (2S)-2-amino-3-(4-(prop-2-ynyloxy)phenyl)propionamide (5.7 mg, 16741 nmol, prepared as described in Example 2) in a buffer consisting of 0.25 mM HEPES and 5 mM EDTA with a pH of 8.0 was prepared. The pH was adjusted to 7.4 by addition of a 4 M aqueous solution of sodium hydroxide (0.0013 ml). A part of this solution (0.005 ml, 4200 nmol) was added to the solution of the FAB-fragment. The pH of the solution was adjusted to 8.03 by addition of a 4 M aqueous solution of sodium hydroxide (0.0003 ml). A solution of CPY in water (200 U/ml, 0.008 ml) was added to the mixture. It was gently shaken at 30° C. for 3 h. A freshly prepared 10 mM solution of phenylmethylsulfonyl fluoride in dry isopropanol (0.0002 ml) was added. The reaction mixture was shaken gently for 30 min at room temperature. Another portion of the mM pheylmethylsulfonyl fluoride solution (0.0053 ml) was added. The reaction mixture was concentrated by centrifugation in a Biomax centrifuge vial with a cut off of 10 000 Da. It was diluted with a 2% solution of 2,6-lutidine in water (0.5 ml). A freshly prepared 100 mM solution of phenylmethylsulfonyl fluoride in dry isopropanol (0.0045 ml) was added. The reaction mixture was concentrated by centrifugation in a Biomax centrifuge vial with a cut off of 10 000 Da to a volume of 0.100 ml. A NAP-5 column was equilibrated with a 2% solution of 2,6-lutidine in water. The solution of the reaction mixture was applied to the column. The protein was washed out with a 2% solution of 2,6-lutidine in water. The solution containing the protein was concentrated by centrifugation in a Biomax centrifuge vial with a cut off of 10 000 Da to a volume of 0.050 ml.

### Step 3: Dimerization of two FAB-Fragments

A solution of copper(II) sulphate (0.36 mg, 1445 nm) in water (0.05 ml) was added to a solution of ascorbic acid (1.30 mg, 7980 nmol) in a mixture of water (0.048 ml) and 2,6-lutidine (0.002 ml). This mixture was left at room temperature for 5 min to form a copper(I) solution.

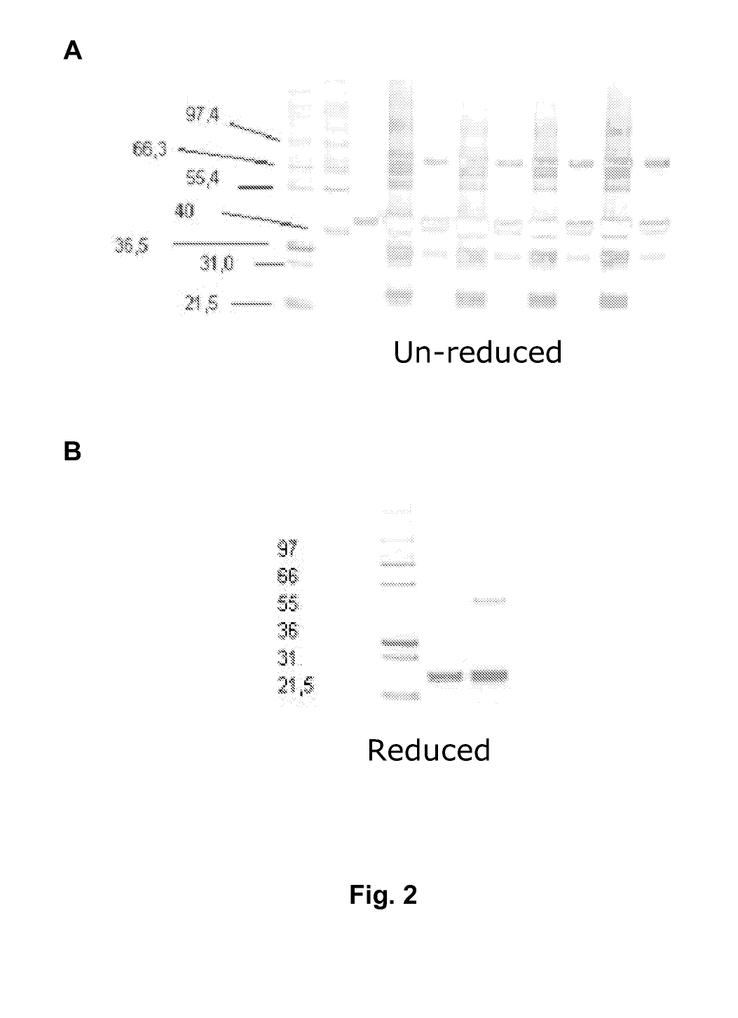

The solutions obtained in Step 1 and in Step 2 were combined. A part of the copper(I) solution (0.010 ml) was added. The reaction mixture was left at room temperature for 3 h. A SDS-gel electrophoreses under non-reducing conditions and a MALDI-TOF analysis were consistent with the expectations for the dimerized product. A second reaction-sequence containing Steps-1-3 was run again. A SDS-gel electorphoresis under reducing conditions was consistent with the expectations (FIG. 2).

### Example 2

### (2S)-2-Amino-3-(4-(prop-2-ynyloxy)phenyl)propionamide

HPLC Method 02-b4-4:

RP-analyses were performed using an Alliance Waters 2695 system fitted with a Waters 2487 dualband detector. UV detections at 214 nm and 254 nm were collected using a Symmetry300 C18, 5 um, 3.9 mm×150 mm column, 42° C. The compounds are eluted with a linear gradient of 5-95% acetonitrile in water which is buffered with 0.05% trifluoroacetic acid over 15 minutes at a flow-rate of 1.0 min/min.

Step 1:

### [1-Carbamoyl-2-(4-hydroxyphenyl)ethyl]-carbamic acid tert-butyl ester

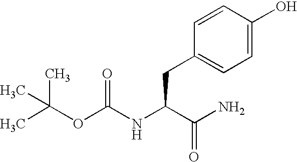

At 0° C., 1-(3-dimethylaminopropyl)-3-ethylcarbodiimide hydrochloride (17.0 g, 88.9 mmol) was added to a solution of (S)-2-(tert-butoxycarbonylamino)-3-(4-hydroxyphenyl)propionic acid (25 g, 88.9 mmol) and 1-hydroxybenzotriazole (12.0 g, 88.9 mmol) in N,N-dimethylformamide (250 ml) and dichloromethane (250 ml). The reaction mixture was stirred at 0° C. for 20 min. A 25% aqueous solution of ammonia in water (90 ml) was added. The reaction mixture was stirred for 3 days at room temperature. It was diluted with ethyl acetate (500 ml) and acidified with a 10% aqueous solution of sodium hydrogensulphate. The phases were separated. The aqueous phase was extracted with ethyl acetate (300 ml). The combined organic layers were washed with a mixture of water (250 ml) and a saturated aqueous solution of sodium hydrogencarbonate solution (250 ml). They were dried over magnesium sulphate. The solvent was removed in vacuo. The crude product was crystallized from ethyl acetate/heptane.

MS: m/z=303 (M+Na)⁺.

¹H-NMR (DMSO-d₆): δ 1.31 (s 9H); 2.80 (dd, 1H); 2.83 (dd, 1H); 4.00 (m, 1H); 6.62 (d, 2H); 6.70 (d, 1H); 6.97 (br, 1H); 7.03 (d, 2H); 7.31 (br, 1H); 9.14 (s, 1H).

Step 2:

### [(S)-1-Carbamoyl-2-(4-(prop-2-ynyloxy)phenyl)ethyl]carbamic acid tert-butyl ester

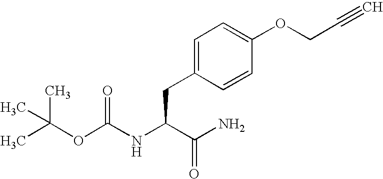

A mixture of [(S)-1-carbamoyl-2-(4-hydroxyphenyl)ethyl]carbamic acid tert-butyl ester (1.0 g, 3.57 mmol), tetrabutylammonium iodide (65 mg, 0.17 mmol), potassium carbonate (3.94 g, 29 mmol), propargyl bromide (0.38 ml, 4.28 mmol) and N,N-dimethylformamide (15 ml) was heated to 60° C. for 16 h. It was cooled to room temperature, diluted with water (30 ml) and acidified with a 10% aqueous solution of sodium hydrogensulphate. The mixture was extracted with ethyl acetate (2×100 ml). The combined organic layers were washed with a saturated aqueous solution of sodium hydrogencarbonate (200 ml) and dried over magnesium sulphate. The solvent was removed in vacuo. The crude product was purified by flash chromatography on silica (100 g), using a mixture of dichloromethane/methanol (10:1) as eluent, to give 998 mg of [(S)-1-carbamoyl-2-(4-(prop-2-ynyloxy)phenyl)ethyl]carbamic acid tert-butyl ester.

MS: m/z=341 (M+Na)⁺.

¹H-NMR (DMSO-d₆) δ 1.31 (s, 9H); 2.50 (s, 1H); 2.67 (dd, 1H); 2.91 (dd, 1H); 4.03 (m, 1H); 4.74 (s, 2H); 6.77 (d, 1H); 6.86 (d, 2H); 6.99 (s, 1H), 7.17 (d, 2H); 7.35 (s, 1H).

Step 3:

### (2S)-2-Amino-3-(4-(prop-2-ynyloxy)phenyl)propionamide

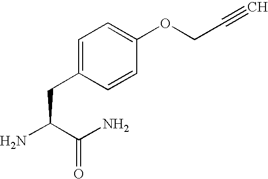

Trifluoroacetic acid (10 ml) was added to a solution of [(S)-1-carbamoyl-2-(4-(prop-2-ynyloxy)phenyl)ethyl]carbamic acid tert-butyl ester (998 mg, 3.13 mmol) in dichloromethane (10 ml). The reaction mixture was stirred for 1.5 h at room temperature. The solvent was removed. The residue was dissolved in dichloromethane (30 ml). The solvent was removed. The latter procedure was repeated twice to give 1.53 g of the trifluoroacetate salt of (2S)-2-amino-3-(4-(prop-2-ynyloxy)phenyl)propionamide.

HPLC (method 02-B4-4): R_{f}=5.62 min.

MS: m/z=219 (M+1)⁺.

¹H-NMR (CDCl₃) δ 2.51 (s, 1H); 3.02 (m, 2H); 3.90 (m, 1H); 4.78 (s, 2H); 6.95 (d, 2H); 7.20 (d, 2H); 7.56 (s, 1H); 7.87 (s, 1H); 8.10 (br, 3H).

### Example 3

### Dimerization of two Fab Fragments and Subsequent Purification

Step 1:

### ((S)-5-(tert-Butoxycarbonylamino)-5-(carbamoyl)pentyl)carbamic acid benzyl ester

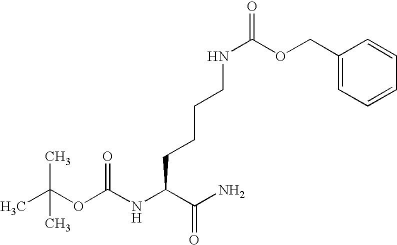

2,5-Dioxopyrrolidin-1-yl(S)-6-((benzyloxycarbonyl)amino)-2-((tertbutoxycarbonyl)amino)hexanoate (commercially available at e.g. Fluke or Bachem, 15. g, 31 mmol) was dissolved in dichloromethane (50 ml). A 25% solution of ammonia in water was added. The reaction mixture was stirred vigorously for 16 h at room temperature. The solvent was removed in vacuo to yield 21.27 g of crude ((S)-5-(tert-butoxycarbonylamino)-5-(carbamoyl)pentyl)carbamic acid benzyl ester, which was used in the next step without further purification.

¹H-NMR (DMSO-d₆): δ 1.2-1.6 (m, 6H); 1.37 (s, 9H); 2.95 (q, 2H); 3.80 (td, 1H); 5.00 (s, 2H); 6.70 (d, 1H); 6.90 (s, 1H); 7.20-7.40 (m, 7H).

MS: m/z=280.

Step 2:

### ((S)-5-Amino-1-(carbamoyl)pentyl)carbamic acid tert-butyl ester

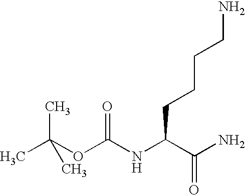

Crude ((S)-5-(tert-butoxycarbonylamino)-5-(carbamoyl)pentyl)carbamic acid benzyl ester (11.92 g, 31.41 mmol) was suspended in methanol (250 ml). Palladium on coal (50% wet) 1.67 g was added. The mixture was subjected to hydrogenation under pressure for 16 h. It was filtered through a plug of celite. The solvent was removed in vacuo to give 13.13 g of crude ((S)-5-amino-1-(carbamoyl)pentyl)carbamic acid tert-butyl ester, which was used in the next step without further purification.

¹H-NMR (DMSO-d₆): δ 1.30-1.60 (m, 6H); 1.37 (s, 9H); 2.65 (t, 2H); 3.80 (dt, 1H); 5.70 (br, 2H); 6.80 (d, 1H); 6.95 (s, 1H); 7.30 (s, 1H).

Step 3:

### Methyl 3-(azidomethyl)benzoate

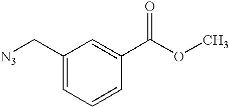

Sodium azide (5.68 g, 87 mmol) was added to a solution of methyl 3-(bromomethyl)benzoate (5.00 g, 22 mmol) in N,N-dimethylformamide (50 ml). Tetrabutylammonium iodide (81 mg, 0.22 mmol) was added. The reaction mixture was heated to 60° C. for 16 h. It was cooled to room temperature and given onto water (200 ml). This mixture was extracted with ethyl acetate (400 ml). The organic layer was washed with water (3×200 ml) and successively dried over sodium sulphate. The solvent was removed in vacuo to give 4.11 go of crude methyl 3-(azidomethyl)benzoate, which was used without further purification.

MS: m/z=192.

¹H-NMR (CDCl₃): δ 3.92 (s, 3H); 4.40 (s, 2H); 7.50 (m, 2H); 8.00 (m, 2H).

Step 4:

### 3-(Azidomethyl)benzoic acid

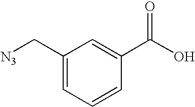

A solution of lithium hydroxide (3.81 g, 21.5 mmol) in water (25 ml) was added to a solution of crude methyl 3-(azidomethyl)benzoate (4.11 g, 21.5 mmol) in 1,4-dioxane (25 ml). Water and 1,4-dioxane was added until a clear solution was obtained. The reaction mixture was stirred for 16 h at room temperature. An 1 N aqueous solution of sodium hydroxide (100 ml) was added. The reaction mixture was washed with tert-butyl methyl ether (2×100 ml). The aqueous phase was acidified with a 10% aqueous solution of sodium hydrogensulphate. It was extracted with ethyl acetate (2×200 ml). The combined ethyl acetate phases were dried over magnesium sulphate. The solvent was removed in vacuo to give 3.68 g of crude 3-(azidomethyl)benzoic acid, which was used without further purification.

MS: m/z=150

¹H-NMR (CDCl₃): δ 4.57 (s, 3H); 7.55 (m, 2H); 8.00 (m, 2H); 13.10 (br, 1H).

Step 5:

### Pyrrolidin-2,5-dione-1-yl 3-(azidomethyl)benzoic ester

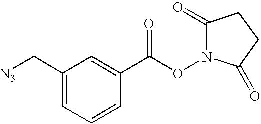

2-Succinimido-1,1,3,3-tetramethyluronium tetrafluoroborate (TSTU, 32.52 g, 107 mmol) was added to a solution of 3-(azidomethyl)benzoic acid (19.01 g, 107 mmol) and triethylamine (14.96 ml, 107 mmol) in N,N-dimethylformamide (50 ml). The reaction mixture was stirred for 16 h at room temperature. It was diluted with ethyl acetate (250 ml) and washed with water (3×120 ml). The organic layer was washed with a saturated aqueous solution of sodium hydrogencarbonate (150 ml) and dried over sodium sulphate. The solvent was removed in vacuo to give 25.22 g of pyrrolidin-2,5-dione-1-yl 3-(azidomethyl)benzoic ester.

¹H-NMR (CDCl₃) δ 2.92 (m, 4H); 4.45 (s, 2H); 7.55 (t, 1H), 7.65 (d, 2H); 8.10 (m, 2H).

Step 6:

### (S)-6-(3-(Aminomethyl)benzoylamino)-2-(tert-butoxycarbonylamino)hexanoic amide

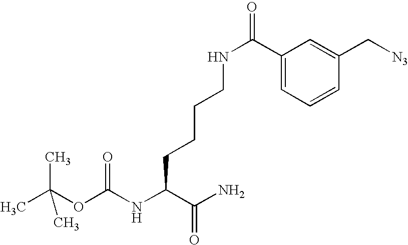

Crude (S)-5-amino-1-(carbamoyl)pentyl)carbamic acid tert-butyl ester (10.26 g, 41.82 mmol) was dissolved in N,N-dimethylformamide (150 ml). Pyrrolidin-2,5-dione-1-yl 3-(azidomethyl)benzoic ester (11.47 g, 41.822 mmol) and ethyldiisopropylamine (21.48 ml, 125.5 mmol) were added successively. The reaction mixture was stirred for 16 h at room temperature. It was diluted with ethyl acetate (500 ml) and washed first with a 10% aqueous solution of sodium hydrogensulphate (200 ml), water (3×250 ml) and a saturated aqueous solution of sodium hydrogencarbonate (200 ml). It was dried over sodium sulphate. The solvent was removed in vacuo to give 6.05 g of (S)-6-(3-(aminomethyl)benzoylamino)-2-(tert-butoxycarbonylamino)hexanoic amide.

¹H-NMR (CDCl₃) δ 1.40 (s, 9H); 1.63 (m, 4H); 1.83 (m, 2H); 3.43 (q, 2H); 4.15 (m, 1H); 4.37 (s, 2H); 5.56 (d, 1H); 6.08 (s, 1H); 6.75 (s, 1H); 7.00 (s, 1H); 7.43 (m, 2H); 7.77 (m, 2H).

MS: m/z=427 (M+Na)⁺, 305 (M-Boc)⁺.

Step 7:

### (S)-2-Amino-6-(3-(azidomethyl)benzoylamino)hexanoic amide

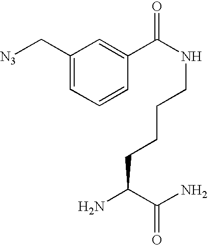

Gaseous hydrogen chloride was bubbled two times for 15 min each through a suspension of (S)-6-(3-(aminomethyl)benzoylamino)-2-(tert-butoxycarbonylamino)hexanoic amide (6.05 g, 14.96 mmol) in ethyl acetate (75 ml). The solvent was removed in vacuo. The crude product was purified by 9 runs of a HPLC-chromatography on a C18-reversed phase column, using a gradient of 8-28% acetonitrile in water, which was buffered with 0.1% trifluoroacetic acid, to give together 5.03 g of the trifluoroacetic acid salt of (S)-2-amino-6-(3-(azidomethyl)benzoylamino)hexanoic amide

HPLC: 6.53 min (method 02-b1-2).

¹H-NMR (DMSO-d₆) δ 1.36 (m, 2H); 1.55 (m, 2H); 1.75 (m, 2H); 3.26 (q, 2H); 3.70 (m, 1H); 4.53 (s, 2H); 7.52 (m, 3H); 7.84 (m, 3H); 8.06 (br, 3H); 8.54 (t, 1H).

MS: m/z=305 (M-F1)⁺

Step 8:

### 2-(Prop-2-ynyloxy)benzoic acid methyl ester

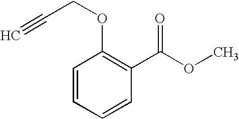

Propargyl bromide (3.116 ml, 36.1 mmol) was added to a mixture of methyl 2-hydroxybenzoate (4.223 ml, 32.9 mmol), potassium carbonate (9.084 g, 65.7 mmol), and tetrabutylammonium iodide (607 mg, 1.64 mmol) in N,N-dimethylformamide (50 ml). The reaction mixture was stirred at 60° C. for 16 h. It was cooled to room temperature. Water was added until all salt was dissolved. The mixture was extracted with ethyl acetate (400 ml). The organic layer was washed with water (3×200 ml) and with a saturated aqueous solution of sodium hydrogencarbonate (200 ml). It was dried over sodium sulphate. The solvent was removed in vacuo. The crude product was purified by flash-chromatography on silica (90 g), using a mixture of ethyl acetate/heptane (1:2) as eluent to give 4.09 g of 2-(prop-2-ynyloxy)benzoic acid methyl ester.

MS: M7z=191, required for M+1: 191.

¹H-NMR (CDCl₃) δ 2.53 (t, 1H); 3.89 (s, 3H); 4.80 (d, 2H); 7.04 (t, 1H); 7.14 (d, 1H); 7.48 (t, 1H); 7.82 (d, 1H).

Step 9:

### 2-(Prop-2-ynyloxy)benzoic acid

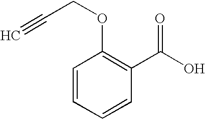

A solution of lithium hydroxide (0.604 g, 25.2 mmol) in water (50 ml) was added to a solution of 2-(prop-2-ynyloxy)benzoic acid methyl ester (4 g, 21.03 mmol) in 1,4-dioxane (50 ml). The reaction mixture was stirred for 16 h at room temperature. It was made basic with an 1 N aqueous sodium hydroxide solution and washed with tert-butyl methyl ether (3×100 ml). The aqueous phase was acidified to pH 2-3 by addition of a 10% aqueous solution of sodium hydrogensulphate. It was extracted with ethyl acetate (3×200 ml). The combined ethyl acetate layers were dried over sodium sulphate. The solvent was removed in vacuo to give 3.07 g of 2-(prop-2-ynyloxy)benzoic acid.

MS: m/z=177, required for M+1: 177.

¹H-NMR (CDCl₃) δ 2.65 (t, 1H); 4.94 (d, 2H); 7.18 (m, 2H); 7.58 (t, 1H); 8.17 (d, 1H); 10.5 (br, 1H).

Step 10:

### 2-(Prop-2-ynyloxy)benzoic acid 2,5-dioxo-pyrrolidin-1-yl ester

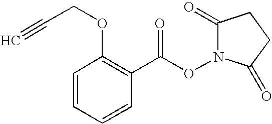

2-Succinimido-1,1,3,3-tetramethyluronium tetrafluoroborate (TSTU, 5.70 g, 18.8 mmol) was added to a solution of 2-(prop-2-ynyloxy)benzoic acid (3.01 g, 17.1 mmol) in N,N-dimethylformamide (50 ml). Ethyldiisopropylamine (7.14 ml, 61.26 mmol) was added. The reaction mixture was stirred for 16 h at room temperature. It was diluted with ethyl acetate (100 ml) and washed with a 10% aqueous solution of sodium hydrogensulphate (200 ml). The aqueous phase was extracted with ethyl acetate (2×200 ml). The combined organic layers were washed with a mixture of water (100 ml) and brine (100 ml) and dried over sodium sulphate. The solvent was removed in vacuo. The crude product was recrystallized from ethyl acetate to give 2.86 g of 2-(prop-2-ynyloxy)benzoic acid 2,5-dioxo-pyrrolidin-1-yl ester.

MS: m/z=296, required for M+Na⁺: 296

¹H-NMR (DMSO-d₆) δ 2.88 (m, 4H); 3.65 (t, 1H); 4.98 (d, 2H); 7.19 (t, 1H); 7.35 (d, 1H); 7.77 (t, 1H); 7.93 (d, 1H).

Step 11:

### [(S)-1-Carbamoyl-5-(2-(prop-2-ynyloxy)benzoylamino)pentyl]carbamic acid tert-butyl ester

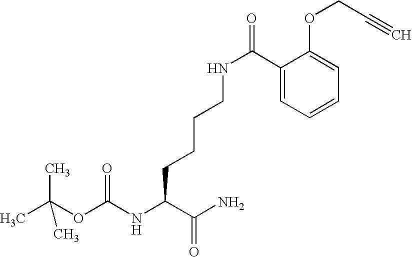

2-(Prop-2-ynyloxy)benzoic acid 2,5-dioxo-pyrrolidin-1-yl ester (2.80 g, 10.25 mmol) was added to a solution of ((S)-5-amino-1-(carbamoyl)pentyl)carbamic acid tert-butyl ester (2.77 g, 11.27 mmol) in N,N-dimethylformamide (50 ml). Ethyldiisopropylamine (4.29 ml, 30.11 mmol) was added. The reaction mixture was stirred at room temperature for 16 h. It was diluted with ethyl acetate (300 ml) and washed with a 10% aqueous solution of sodium hydrogensulphate. The aqueous phase was extracted with ethyl acetate (2×100 ml). The combined organic layers were washed with a mixture of brine (100 ml) and water (100 ml) and subsequently with a saturated aqueous solution of sodium hydrogencarbonate. They were dried over sodium sulphate. The solvent was removed in vacuo to give 3.99 g of crude [(S)-1-carbamoyl-5-(2-(prop-2-ynyloxy)benzoylamino)pentyl]carbamic acid tert-butyl ester, which was used for the next step without further purification.

MS: m/z=404, required for M+1: 404

¹H-NMR (DMSO-d₆) δ 1.30-1.80 (m, 6H); 1.37 (s, 9H); 3.24 (q, 2H); 3.63 (t, 1H); 3.85 (m, 1H); 4.92 (d, 2H); 6.73 (d, 1H); 6.93 (br, 1H); 7.05 (t, 1H); 7.18 (d, 1H); 7.23 (br, 1H); 7.45 (t, 1H); 7.68 (d, 1H); 8.10 (t, 1H).

Step 12:

### N—((S)-5-Amino-5-carbamoylpentyl)-2-(prop-2-ynyloxy)benzamide

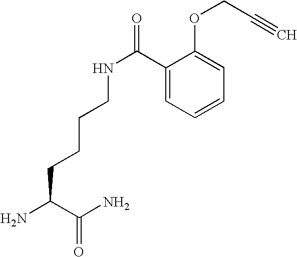

Trifluoroacetic acid (50 ml) was added to a solution of [(S)-1-carbamoyl-5-(2-(prop-2-ynyloxy)benzoylamino)pentyl]carbamic acid tert-butyl ester (1.72 g, 4.26 mmol) in dichloromethane (50 ml). The solvent was removed in vacuo. The crude product was purified by HPLC-chromatography on a reversed phase C18-column, using a gradient of 10-30% acetonitrile in water, which was buffered with 0.1% trifluoroacetic acid. The solvent was removed in vacuo. The residue was dissolved in water (20 ml) and lyophilized to give 980 mg of N—((S)-5-amino-5-carbamoyl pentyl)-2-(prop-2-ynyloxy)benzamide.

MS: m/z=304, required for M+1: 304.

HPLC: Rt=4.11 min (method 02-b4-4).

¹H-NMR (DMSO-d₆) δ 1.37 (m, 2H); 1.53 (m, 2H), 1.75 (m, 2H); 3.26 (q, 2H); 3.64 (s, 1H); 3.71 (q, 1H); 4.93 (s, 2H); 7.06 (t, 1H); 7.21 (d, 1H); 7.46 (t, 1H); 7.57 (br, 1H); 7.67 (d, 1H); 7.69 (br, 1H); 8.10 (br, 3H).

Step 13:

Transpeptidation Reaction of a FAB-fragment with (S)-2-Amino-6-(3-(azidomethyl)benzoylamino)hexanoic Amide under Catalysis of CPY

A solution of a FAB-fragment, which at its C-terminus was extended with a leucylleucylalanine-sequence with a concentration of 3.2 mg/ml (0.63 ml, 2 mg, 41 nmol) was transferred into a Biomax filter device (Millipore) with a cut-off of 5 kDa. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 7.96 was added. The solution was concentrated by ultracentrifugation at 12000 G for 6 min. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 8.11 was added. The solution was concentrated by ultracentrifugation at 12000 G for 6 min. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 8.11 (1.00 ml) was added. The solution was concentrated by ultracentrifugation at 12000 G for 6 min. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 8.11 (1.00 ml) was added. The solution was concentrated by ultracentrifugation at 12000 G for 6 min. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 8.11 (1.00 ml) was added. The solution was concentrated by ultracentrifugation at 12000 G for 6 min. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 8.11 (1.00 ml) was added. The solution was concentrated by ultracentrifugation at 12000 G for 6 min. An 150 mM solution of (S)-2-amino-6-(3-(azidomethyl)benzoylamino)hexanoic amide (0.220 ml) in a buffer, consisting of 0.25 M HEPES and 5 mM EDTA, which had been adjusted to pH 8.11 (1.00 ml) was added. The solution was concentrated by ultracentrifugation at 12000 G for 6 min. After this the volume of the mixture was 0.44 ml. A solution of carboxypeptidase Y (200 U/ml, 0.014 ml, 2.6 U) was added. The solution was gently shaken at room temperature for 2.75 h. A freshly prepared 100 mM solution of phenylmethanesulfonyl fluoride in isopropanol (0.004 ml) was added. The reaction mixture was shaken gently for 30 min. It was transferred into a Biomax-filter with a cut-off of 5 kDa. A 2% solution of 2,6-lutidine in water (0.100 ml) was added. A freshly prepared 100 mM solution of phenylmethansulfonyl fluoride in isopropanol (0.001 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. The residue was applied to a NAP™ 5 column (GE Healthcare Uppsala), and the protein was eluted with a 2% solution of 2,6-lutidine in water (1 ml) to give a solution of a FAB-fragment, bearing an azide at its C-terminus.

Step 14:

### Transpeptidation Reaction of a FAB-fragment with N—((S)-5-Amino-5-carbamoylpentyl)-2-(prop-2-ynyloxy)benzamide under Catalysis of CPY

A solution of a FAB-fragment, which at its C-terminus was extended with a leucylleucylalanine-sequence with a concentration of 3.2 mg/ml (0.63 ml, 2 mg, 41 nmol) was transferred into a Biomax filter device with a cut-off of 5 kDa and was concentrated at 12000 G for 6 min. A 150 mM solution of N—((S)-5-amino-5-carbamoylpentyl)-2-(prop-2-ynyloxy)benzamide (0.40 ml) in a buffer consisting of 0.25 M HEPES and 5 mM EDTA, which had been adjusted to pH 8.04 was added. The solution was concentrated at 12000 G for 6 min. A 150 mM solution of N—((S)-5-amino-5-carbamoylpentyl)-2-(prop-2-ynyloxy)benzamide (0.40 ml) in a buffer consisting of 0.25 M HEPES and 5 mM EDTA, which had been adjusted to pH 8.04 was added. The solution was concentrated at 12000 G for 6 min. A 150 mM solution of N—((S)-5-amino-5-carbamoylpentyl)-2-(prop-2-ynyloxy)benzamide (0.20 ml) in a buffer consisting of 0.25 M HEPES and 5 mM EDTA, which had been adjusted to pH 8.04 was added. The solution was concentrated at 12000 G for 6 min. A 150 mM solution of N—((S)-5-amino-5-carbamoylpentyl)-2-(prop-2-ynyloxy)benzamide (0.20 ml) in a buffer consisting of 0.25 M HEPES and 5 mM EDTA, which had been adjusted to pH 8.04 was added. The solution was concentrated at 12000 G for 6 min. A 150 mM solution of N—((S)-5-amino-5-carbamoylpentyl)-2-(prop-2-ynyloxy)benzamide (0.20 ml) in a buffer consisting of 0.25 M HEPES and 5 mM EDTA, which had been adjusted to pH 8.04 was added. The solution was concentrated at 12000 G for 6 min. A 150 mM solution of N—((S)-5-amino-5-carbamoylpentyl)-2-(prop-2-ynyloxy)benzamide (0.20 ml) in a buffer consisting of 0.25 M HEPES and 5 mM EDTA, which had been adjusted to pH 8.04 was added. The solution was concentrated at 12000 G for 6 min resulting in a volume of 0.420 ml. A solution of CPY (200 U/ml, 0.013 ml, 2.5 U) was added to the mixture. It was gently shaken at room temperature for 2.5 h. A freshly prepared 100 mM solution of phenylmethanesulfonyl fluoride in isopropanol (0.004 ml) was added. The reaction mixture was shaken gently for 30 min. It was transferred into a Biomax-filter with a cut-off of 5 kDa. A 2% solution of 2,6-lutidine in water (0.100 ml) was added. A freshly prepared 100 mM solution of phenylmethansulfonyl fluoride in isopropanol (0.001 ml) was added. The mixture was concentrated at 12000 G for 6 min. A 2% solution of 2,6-lutidine in water (0.100 ml) was added. A freshly prepared 100 mM solution of phenylmethansulfonyl fluoride in isopropanol (0.001 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. A 2% solution of 2,6-lutidine in water (0.200 ml) was added. A freshly prepared 100 mM solution of phenylmethansulfonyl fluoride in isopropanol (0.002 ml) was added. The mixture was concentrated at 12000 G for 6 min. The residue was applied to a NAP™ 5 column (GE Healthcare Uppsala), and the protein was eluted with a 2% solution of 2,6-lutidine in water (1 ml) to give a solution of a FAB-fragment, bearing an alkyne at its C-terminus.

Step 15:

The solution obtained in step 13 and the solution obtained in step 14 were combined in a Biomax centrifugal filter device (Millipore) with a cut-off of 5 kDa. The solution was concentrated at 12000 G for 6 min to a volume of approx. 0.30 ml. A 2% solution of 2,6-lutidine in water (0.300 ml) was added. The solution was concentrated at 12000 G for 6 min. A 2% solution of 2,6-lutidine in water (0.300 ml) was added. The solution was concentrated at 12000 G for 6 min. A 2% solution of 2,6-lutidine in water (0.300 ml) was added. The solution was concentrated at 12000 G for 6 min. The mixture was transferred into an Eppendorf vial. A solution of copper sulphate pentahydrate (10.2 mg, 0.041 mmol) in water was added to a solution of ascorbic acid (36 mg, 0.20 mmol) in a mixture of water (0.196 ml) and 2,6-lutidine (0.004 ml) to form a Cu(I) solution. This solution was left for 2 min at room temperature. A part of it (0.004 ml) was added to the solution of proteins. This solution was gently shaken for 3.5 h at room temperature. The mixture was transferred to a Biomax centrifugation device with a cut-off of 5 kDa. A buffer, consisting of 10 mM MES and 200 mM sodium chloride, which had been adjusted to pH 5.5 (0.200 ml) was added. The solution was concentrated at 12000 G for 6 min. A buffer, consisting of 10 mM MES and 200 mM sodium chloride, which had been adjusted to pH 5.5 (0.400 ml) was added. The solution was concentrated at 12000 G for 6 min. A buffer, consisting of 10 mM MES and 200 mM sodium chloride, which had been adjusted to pH 5.5 (0.400 ml) was added. The solution was concentrated at 12000 G for 6 min. A buffer, consisting of 10 mM MES and 200 mM sodium chloride, which had been adjusted to pH 5.5 (0.900 ml) was added to obtain a total volume of 1 ml. The mixture was transferred to a Biomax centrifugation device with a cut-off of 5 kDa and was concentrated at 12000 rcf for 6 min. It was subjected to a gel-chromatography on a Superdex 200 on a 16/60 column. It was eluted from the column with a flow of 1 ml/min, using a buffer of 10 mM MES and 200 mM sodium chloride, which had been adjusted to pH 5.5 as eluent. The fractions containing the desired compound were combined and concentrated in an Amicon Ultra centrifugal filter device (Millipore) with a cut-off of 5 kDa at 4000 rpm for 28 min. The remaining solution (0.600 ml) was analyzed. A concentration of protein was found on a NanoDrop NP1000 Spectrophotometer with an absorption coefficient of 14.11 to be 0.020 mg of the isolated dimerized FAB-fragment to be 350 nM in a purity of approximately 60% based on a SDS-gel, the impurity being monomeric FAB-fragments in an amount of approximately 40%. A further purification of the dimerized FAB-fragment by gel-chromatography on a Superdex 200 16/60 column, using a buffer, consisting of 10 mM MES and 200 mM sodium chloride, which had been adjusted to pH 5.5, at a flow of 0.7 ml/min was not successful, due to the high dilution of the FAB-fragment the starting solution.

### Exemplary Embodiments

The following are exemplary embodiments of the invention.
1. A compound being a dimer of two antibody fragments, wherein said antibody fragments are coupled at their C-termini of the heavy chain (HC) polypeptides.
2. The compound according to embodiment 1, wherein said HC polypeptides are coupled by a non-peptide bond.
3. The compound according to any of embodiments 1-2, wherein the C-terminus of a first HC polypeptide has the structure of

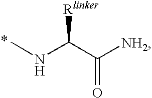

wherein the first polypeptide is marked with “*”, and a second HC-polypeptide is attached to the group R^{linker}.
4. The compound according to embodiment 3, wherein the C-terminus of the first HC-polypeptide has the structure of

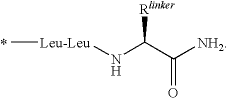

5. The compound according to any of the preceding embodiments, wherein said HC polypeptides are coupled by a reaction between an azide on one of HC polypeptides and an alkyne on the other HC polypeptide.
6. The compound according to any of embodiments 1-4, wherein said HC polypeptides are coupled by a reaction between an O-alkylated hydroxylamine on one of the HC polypeptides and a ketone or an aldehyde on the other HC polypeptide.
7. An antibody fragment wherein the C-terminus of a HC-polypeptide has the structure of

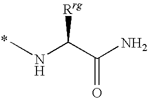

wherein the HC polypeptide is marked with “*” and R^{rg} is a group bearing a group selected from azide, alkyne, O-alkylated hydroxylamine, ketone, aldehyde, 1,2-diol, or 1,2 aminoalcohol.
8. The antibody fragment of embodiment 7, wherein the C-terminus of the HC-polypeptide has the structure of

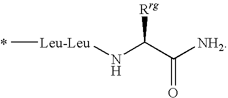

9. The antibody fragment of any of embodiments 7-8, wherein —R^{rg} is selected from

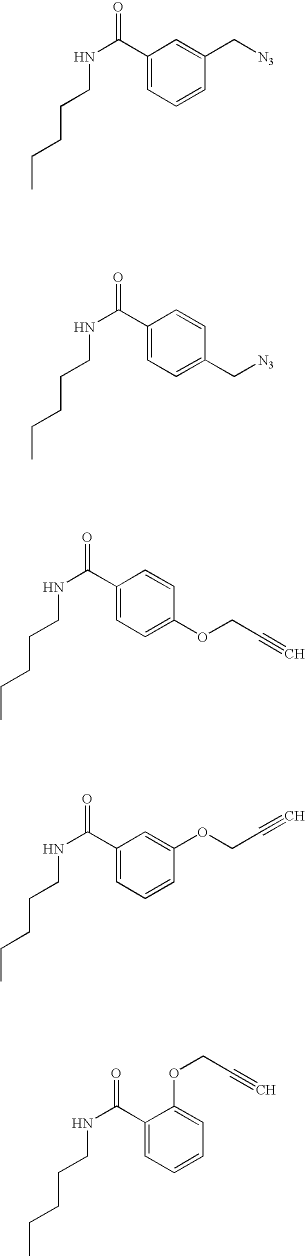

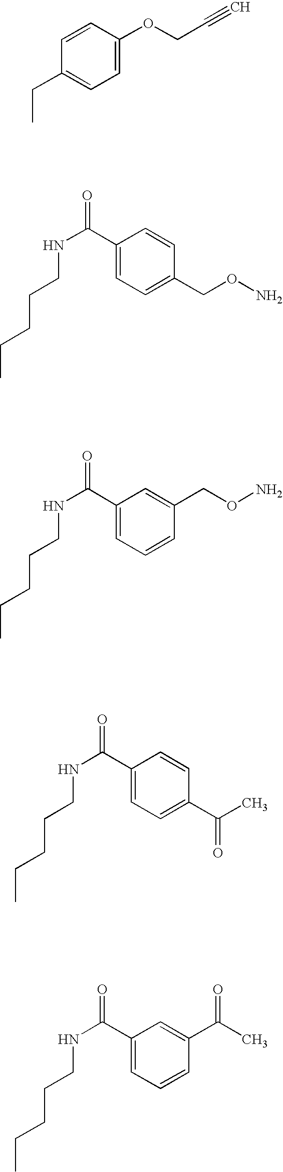

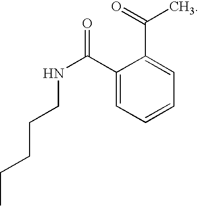

10. A process for dimerization of two antibody fragments comprising the steps of
   (a) introducing a first chemical group to the C-terminus of a HC polypeptide of the first antibody fragment,
   (b) introducing a second chemical group to the C-terminus of a HC polypeptide of the second antibody fragment, and
   (c) reacting the first chemical group with the second chemical group to form a covalent linkage of the two antibody fragments.
11. The process according to embodiment 10, comprising the steps of
   (a′) introducing a first chemical group to the C-terminus of the HC polypeptide of the first antibody fragment by reaction with an enzyme in the presence of a nucleophile,
   (b′) introducing a second chemical group to the C-terminus of the HC polypeptide of the second antibody fragment by reaction with an enzyme in the presence of a nucleophile, and
   (c′) reacting the first chemical group with the second chemical group to form a covalent linkage of the two antibody fragments.
12. The process according to embodiment 10, comprising the steps of
   (a″) introducing a first chemical group to the C-terminus of the HC polypeptide of the first antibody fragment by reaction with an enzyme in the presence of a nucleophile,
   (b″) introducing a third chemical group to the C-terminus of the HC polypeptide of the second antibody fragment by reaction with an enzyme in the presence of a nucleophile,
   (b′″) reacting the third chemical group with a molecule bearing a fourth and a second chemical group to attach said molecule covalently to the C-terminus of the HC polypeptide of the second antibody fragment by reaction of the third chemical group and the fourth chemical group, and
   (c) reacting the first chemical group with the second chemical group to form a covalent linkage of the two antibody fragments.
13. The process according to any of embodiments 10-12, wherein said first chemical group and said second chemical group are different from each other.
14. The process according to any of embodiments 10-13, wherein said chemical groups are separately selected from the group consisting of alkyne, azide, O-alkylated hydroxylamine, ketone, aldehyde, hydrazone and O-acylated hydroxylamine.
15. The process according to any of embodiments 10-14, wherein a reaction between an azide and an alkyne is used to form the linkage between the two antibody fragments.
16. The process according to embodiment 15, wherein said reaction between an azide and an alkyne is catalyzed by copper(I)-ions.
17. The process according to any of embodiments 10-14, wherein a reaction between an O-alkylated hydroxylamine and a ketone or an aldehyde is used to form the linkage between the two antibody fragments.
18. The process according to any of embodiments 10-17, wherein said enzyme is a serine-carboxypeptidase.
19. The process according to any of embodiments 10-18, wherein said enzyme is carboxypeptidase Y.
20. The process according to any of embodiments 10-19, wherein the C-terminal amino acid sequence of at least one of the two HC polypeptides is -Ala.
21. The process according to any of embodiments 10-20, wherein the C-terminal amino acid sequence of at least one of the two HC polypeptides is Leu-Leu-Ala.
22. The process according to any of embodiments 10-21, wherein said two antibody fragments are different from each other.
23. The process according to any of embodiments 11-22, wherein said nucleophile is selected from the group consisting of

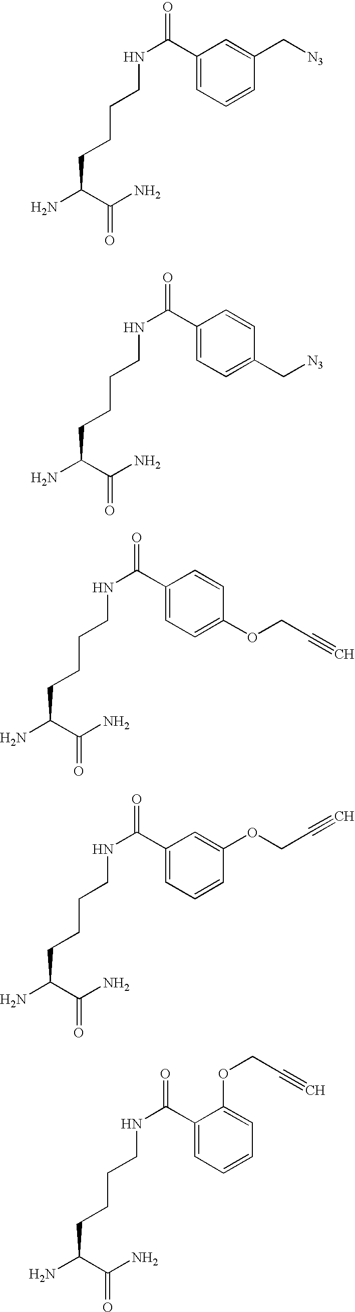

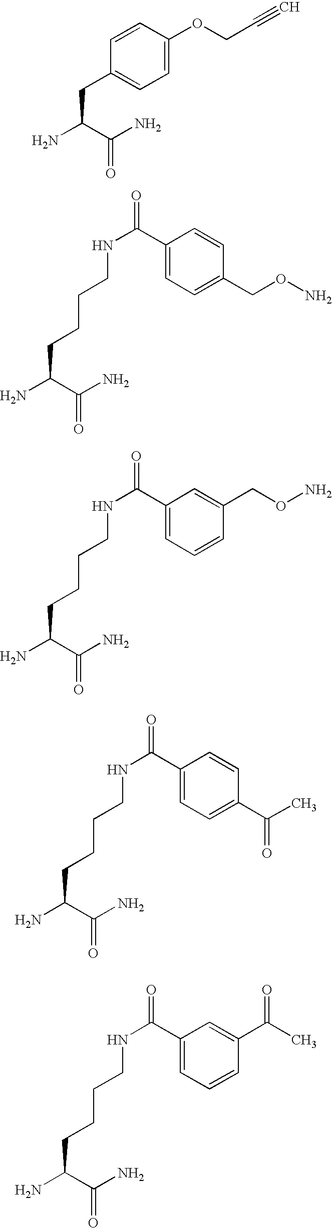

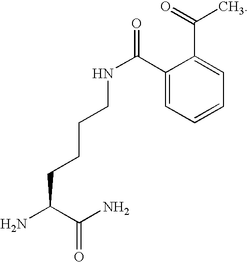

24. The process according to any of embodiments 10-23, wherein said reaction in step (c) forms an 1,2,3-triazole.
25. The process according to any of embodiments 10-24, wherein said reaction in step (c) forms an oxime or a hydrazone.
26. A compound being a dimer of two antibody fragments, said compound being obtainable by the process according to any of embodiments 10-25.
27. An antibody fragment wherein the C-terminal amino acid sequence is Leu-Leu-Ala.

1. A compound being a dimer of two antibody fragments, wherein said antibody fragments are coupled at their C-termini of the heavy chain (HC) polypeptides.

2. The compound according to claim 1, wherein the C-terminus of a first HC polypeptide has the structure of 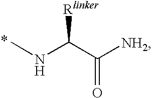
wherein the first polypeptide is marked with “*”, and a second HC-polypeptide is attached to the group R^{linker}.

3. The compound according to claim 2, wherein the C-terminus of the first HC-polypeptide has the structure of

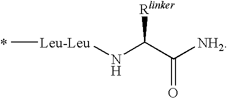

4. An antibody fragment wherein the C-terminus of a HC-polypeptide has the structure of 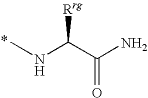
wherein the HC polypeptide is marked with “*” and R^{rg} is a group bearing a group selected from azide, alkyne, O-alkylated hydroxylamine, ketone, aldehyde, 1,2-diol, or 1,2 aminoalcohol.

5. The antibody fragment of claim 4, wherein the C-terminus of the HC-polypeptide has the structure of

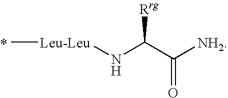

6. The antibody fragment of claim 4, wherein —R^{rg} is selected from

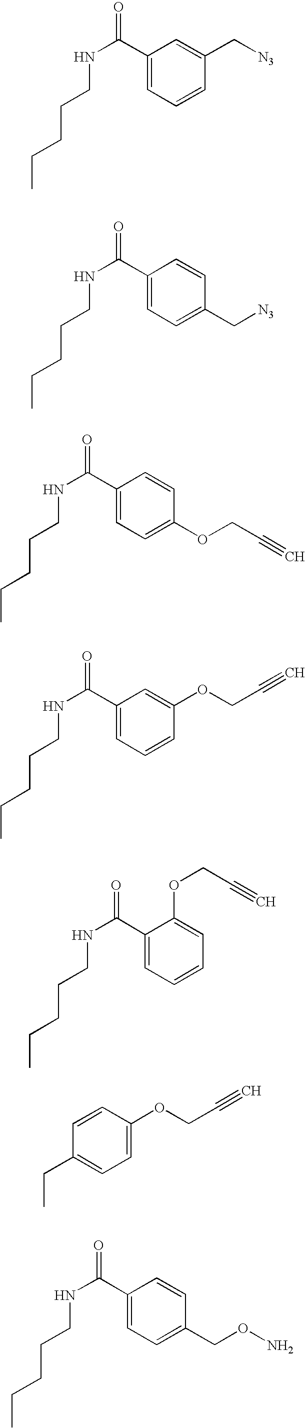

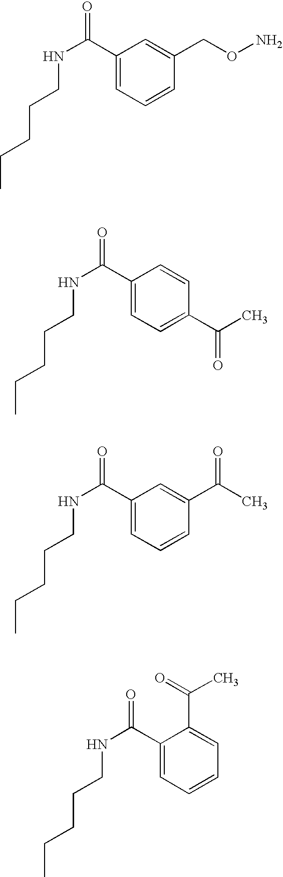

7. A process for dimerization of two antibody fragments comprising the steps of
(a) introducing a first chemical group to the C-terminus of a HC polypeptide of the first antibody fragment,
(b) introducing a second chemical group to the C-terminus of a HC polypeptide of the second antibody fragment, and
(c) reacting the first chemical group with the second chemical group to form a covalent linkage of the two antibody fragments.

8. The process according to claim 7, comprising the steps of
(a′) introducing a first chemical group to the C-terminus of the HC polypeptide of the first antibody fragment by reaction with an enzyme in the presence of a nucleophile,
(b′) introducing a second chemical group to the C-terminus of the HC polypeptide of the second antibody fragment by reaction with an enzyme in the presence of a nucleophile, and
(c′) reacting the first chemical group with the second chemical group to form a covalent linkage of the two antibody fragments.

9. The process according to claim 7, comprising the steps of
(a″) introducing a first chemical group to the C-terminus of the HC polypeptide of the first antibody fragment by reaction with an enzyme in the presence of a nucleophile,
(b″) introducing a third chemical group to the C-terminus of the HC polypeptide of the second antibody fragment by reaction with an enzyme in the presence of a nucleophile,
(b′″) reacting the third chemical group with a molecule bearing a fourth and a second chemical group to attach said molecule covalently to the C-terminus of the HC polypeptide of the second antibody fragment by reaction of the third chemical group and the fourth chemical group, and
(c) reacting the first chemical group with the second chemical group to form a covalent linkage of the two antibody fragments.

10. The process according to claim 7, wherein said first chemical group and said second chemical group are different from each other.

11. The process according to claim 7, wherein a reaction between an azide and an alkyne is used to form the linkage between the two antibody fragments, and said reaction between an azide and an alkyne is catalyzed by copper(I)-ions.

12. The process according to claim 7, wherein a reaction between an O-alkylated hydroxylamine and a ketone or an aldehyde is used to form the linkage between the two antibody fragments.

13. The process according to claim 7, wherein said enzyme is carboxypeptidase Y.

14. The process according to claim 7, wherein the C-terminal amino acid sequence of at least one of the two HC polypeptides is -Leu-Leu-Ala.

15. The process according to claim 8, wherein said nucleophile is selected from the group consisting of 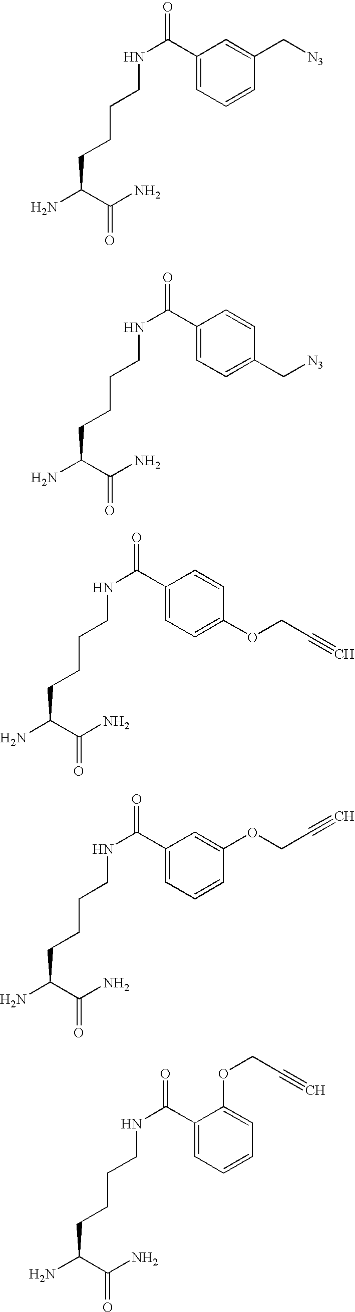 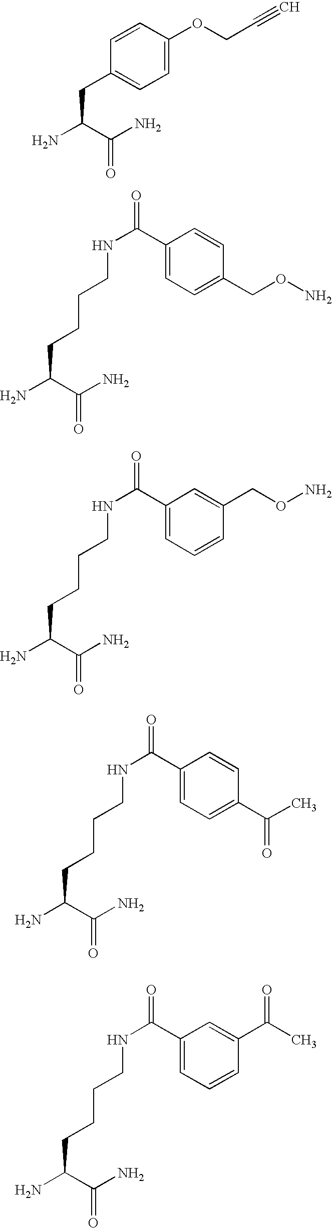 [chemical structure]
